(19) United States
(12) Patent Application Publication
(10) Pub. No.: US 2003/0007961 A1
(43) Pub. Date: Jan. 9, 2003

(54) Orthomolecular vitamin E derivatives

(76) Inventor: Michael D. Wilburn, Cedar Hill, TX (US)

Correspondence Address: NATH & ASSOCIATES, 1030 15th STREET, 6TH FLOOR, WASHINGTON, DC, 20005, US

(21) Appl. No.: 09/886,472

(22) Filed: Jun. 22, 2001

(51) Int. Cl. A61K 38/44; A61K 31/726; A61K 31/714

(52) U.S. Cl. 424/94.4; 514/46; 514/54; 514/100; 514/52; 514/251; 514/336; 514/27; 514/458; 435/189; 536/8; 536/26.13; 536/27.3; 536/53; 544/257; 546/282.7; 549/406; 549/408

(57) ABSTRACT

Orthomolecular Vitamin E derivative compounds, compositions, and their uses for effecting aging and longevity, nerve activity, hematopoiesis and maintenance of blood cells, hepatic activity, nephritic activity, heart and cardiovascular function, pulmonary function, muscular function, cartilage, bone, and joint health, gastrointestinal function, reproductive system function, vision, immune function, cell membrane integrity, and pain and inflammation; preventing or treating diseases or conditions; treating cancers or obesity; and reducing the risk of Sudden Infant Death Syndrome in an animal. The compounds of the present invention are of formula I:

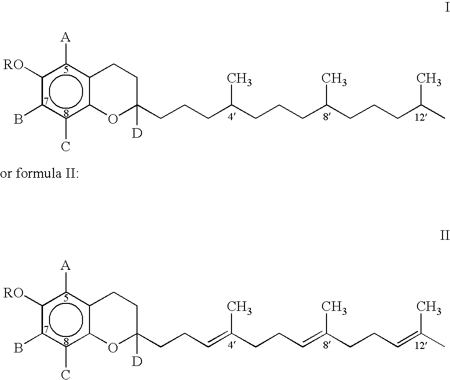

or a pharmaceutically acceptable salt, ester, or solvate, thereof, wherein:

A, B, C, D, and R are as defined herein.

### BACKGROUND OF THE INVENTION

1. Field of Invention

The present invention relates to novel vitamin E derivative compounds and pharmaceutical preparations which are useful in effecting a biological activity in an animal, such as aging and longevity, nerve activity, hematopoiesis and maintenance of blood cells, hepatic activity, nephritic activity, heart and cardiovascular function, pulmonary function, muscular function, cartilage, bone, and joint health, gastrointestinal function, reproductive system function, vision, immune function, cell membrane integrity, and pain and inflammation; in preventing or treating diseases or conditions; in treating cancers or obesity; and in reducing the risk of Sudden Infant Death Syndrome. 2. Background

Linus Pauling coined the term “Orthomolecular Medicine” and defined it as: “The preservation of good health and the prevention and treatment of disease by varying the concentrations in the human body of the molecules or substances that are normally present, many of them required for life, such as the vitamins, essential amino acids, essential fats, and minerals.” Literally, the term is derived from the Greek “ortho”, for correct or right, and “molecule”, or “right molecule”. When these “right molecules” are out of balance, disorders and disease can result. The Orthomolecular Concomitant Theory of Convergence suggests that the duality of stress and uncontrolled free radical proliferation are major disruptive forces in the delicate balance of life, resulting in disorders, diseases, and premature death. In particular, the free radical theory of aging and disease suggests that excess free radicals can be generated by simple aging or exposure to toxic pollutants in air, water, and foods, as well as cigarette smoke, alcohol, and ionizing radiation. Free radicals may produce oxidative damage to DNA and other cell components which accumulates with age and is suggested to be a major contributor to aging and degenerative diseases. Antioxidants may be used for reducing, eliminating, preventing, and reversing oxidative damage to tissues in an animal. Treatment of disorders and disease by orthomolecular methods is aimed at bringing such natural substances into healthful balance.

In a relatively recent phenomenon, traditional primary health care practitioners have begun to embrace orthomolecular nutrition as an enhancement to their practices. There are several forces promoting this trend, including consumer demand and the increasing eligibility of alternative health care for medical insurance coverage.

The National Institutes of Health (NIH) has established the National Center for Complementary and Alternative Medicine (NCCAM) to assist in prioritizing applications for research grants in complementary and alternative medicine (CAM). The NCCAM classification system is divided into seven major categories and includes examples of practices or preparations in each category. The biologically-based therapies category includes natural and biologically-based practices, interventions, and products. One subcategory is orthomolecular medicine, which refers to products used as nutritional and food supplements for preventive or therapeutic purposes. The NCCAM classification system lists ascorbic acid, carotenes, tocopherols, folic acid, niacin, niacinamide, pantothenic acid, pyridoxine, riboflavin, thiamine, vitamin A, vitamin D, vitamin K, biotin, choline, s-adenosylmethionine, calcium, magnesium, selenium, potassium, taurine, lysine, tyrosine, gamma-oryzanol, iodine, iron, manganese, molybdenum, boron, silicon, vanadium, co-enzyme Q₁₀, carnitine, probiotics, glutamine, phenylalanine, glucosamine sulfate, chondroitin sulfate, lipoic acid, amino acids, phosphatidylserine, melatonin, DHEA, inositol, glandular products, fatty acids, and medium chain triglycerides as examples of orthomolecular substances. Other examples of orthomolecular substances include omega-3 fatty acids, lycopene, soy isoflavonoids, tocotrienols, chromium, zinc, and copper.

Vitamin E is an important compound that occurs in many living cells and takes part in several biological processes in the human body. For example, U.S. Pat. No. 5,919,818 (Jul. 6, 1999) claims novel tocotrienols, tocotrienol-like compounds, and mixtures, as well as the use of thereof as hypocholesterolemic, antithrombotic, antioxidizing, antiatherogenic, antiinflammatory, and immunoregulatory agents, or as agents useful to decrease lipoprotein (a) concentration in the blood or to increase feed conversion efficiency. The term vitamin E applies to a family of eight related compounds, the tocopherols and the tocotrienols. The four major forms of vitamin E are designated α, β, γ, and δ, on the basis of the chemical structure. The tocotrienols are less widely distributed in nature than the tocopherols, although they are present in various oils such as palm oil. α-tocopherol is the most potent and most commonly used form of this fat-soluble nutrient. Tocotrienols may have biological activity comparable with that of the tocopherols but were once considered of less nutritional importance. α-tocopherol, commonly known as vitamin E, is the form found most in nature and the most biologically active.

Foods high in vitamin E include wheat germ, whole grains, cold-pressed vegetable oils, nuts and seeds, dark green leafy vegetables, eggs, sweet potatoes, brussels sprouts and whole wheat. Vitamin E supplements are available in both dry form and oil capsules. Vitamin E is also available in the natural D-α-tocopherol form and in the synthetic DL-α-tocopherol form.

Tocopherol and tocotrienol compounds may be recovered from many biological materials including, but not limited to, oats, wheat, rye, barley, soybean, wheat germ, wheat bran, corn, rice, cottonseed, milkweed, flax, sesame, rice bran, parboiled brown rice, brown rice flour, olives, vegetable oil distillant, fruit concentrate evaporate, barley bran, palm oil, wheat germ oil, rice bran oil, barley oil, coconut oil, cottonseed oil, soybean oil, other cereal grains and other cereal grain oils, plant tissues, flowers, juniper and other bushes, various trees, various fruits, leafy vegetables, alfalfa and other grasses, mushrooms and other fungi, leaves, seeds, stems, bark, roots, nuts, and legumes.

Scientists are now discovering that all members of the vitamin E family have important functions, such as the role of γ-tocopherol and tocotrienols. γ-tocopherol scavenges nitrogen free radicals. These radicals are involved in arthritis, multiple sclerosis (MS) and diseases of the brain (such as Alzheimer's). A metabolic product appears to help regulate the amount of fluid and electrolytes that pass through the kidney and end up in urine. Thus, γ-tocopherol may play a significant role in blood pressure control, and treating congestive heart failure and cirrhosis of the liver.

Tocotrienols also slow the narrowing of the carotid artery and reduce total cholesterol, LDL, and triglycerides. Tocotrienols may also slow the growth of breast, colon, and leukemia cancer. In another embodiment of this invention, tocopherol and tocotrienol compounds may be administered pre-operatively to a patient in order to prevent septic shock.

Hypercholesterolemia involves high serum cholesterol levels and is a causative agent of diseases including arteriosclerosis, atherosclerosis, cardiovascular disease, and xanthomatosis. In addition, high serum cholesterol levels are seen in patients suffering from diseases including diabetes mellitus, familial hypercholesterolemia, acute intermittent prothyria, anorexia nervosa, nephritic syndrome, primary cirrhosis and various liver disorders, such as hepatitis and obstructive jaundice. Improvement of lipoprotein profiles and a decrease in total serum and low density lipoprotein cholesterol have been shown to retard the progression of such diseases, as well as to induce regression of clinically significant lesions in hypercholesterolemic patients. α-tocotrienol, a chromanol isolated from barley extract, has been identified as a therapeutic agent for hypercholesterolemia. In addition, α-tocotrienol, γ-tocotrienol, and δ-tocotrienol have also been shown to reduce hypercholesterolemia.

Although the relationship between hypercholesterolemia and its many associated diseases, most notably cardiovascular disease, has been extensively studied, no clear answer to this world-wide problem has yet been found. As a result, coronary artery disease remains the leading cause of death in the United States and other developed countries. Coronary artery disease is the result of complex interactions between a large number of different processes, including lipoprotein metabolism, aggregation of blood platelets, blood coagulation and fibrinolysis. Accordingly, the cardiovascular risk profile of a given patient is dependent on these interactions.

In addition to lowering cholesterol levels, the cardiovascular risk profile of a patient may also be reduced by decreasing the levels of other factors in the serum and the blood. For example, reduction of thromboxane A₂ generation (measured by the levels of thromboxane B₂, a stable metabolite of thromboxane A₂), and platelet factor 4 levels in the serum lessens the risk of cardiovascular disease because of decreased thrombogenic activity.

Thromboxane A₂ and platelet factor 4 levels are also associated with other biological activities. For example, when reduction of these factors is accompanied by a reduction in macrophage cell count, lower tumor necrosis factor (TNF) levels, and lower arachidonic acid levels in bodily tissues, reduced levels of prostaglandins, leukotrienes, and interleukins are implicated. Reduction of these factors, therefore, leads to a decrease in the inflammation accompanying a wide variety of diseases. In addition, since prostaglandins inhibit glucose-induced insulin release and increase glucagon secretion, an increased insulin to glucagon ratio may also result from the reduction in prostaglandins. Such an increase is useful in improving glucose intolerance in diabetes mellitus and restoration of acute glucose-induced insulin response in non-insulin-dependent diabetes mellitus.

It has been noted that there is a low incidence of cardiovascular disease in populations consuming large amounts of cereal grains. Soluble and insoluble fibers have, in the past, been viewed as the agents responsible for cholesterol reduction in such populations.

More recently, it has been suggested that high levels of homocysteine are strongly associated with arteriosclerosis, independent of other risk factors. Controlling metabolic levels of homocysteine through orthomolecular methods may reduce or eliminate some risks of heart disease and stroke (see Cooney and Lawren, Methyl Magic: Maximum Health Through Methylation, Andrews McMeel Publishing, 1999, Chap. 10).

As a class, the tocopherols, including d-α-tocopherol, have been extensively studied. As a result of these studies, certain biological activities have been attributed to the tocopherols. Such activities include platelet aggregation and antioxidant functions. Although the exact structure-function relationship is not known, several experiments have highlighted the importance of the phytyl side chain in the biological activity of tocopherols.

In contrast to the tocopherols, interest in the tocotrienols has been limited, as those compounds were not typically considered to be biologically useful. Recently, however, studies have indicated that tocotrienols may be biologically active. For example, U.S. Pat. No. 4,603,142 identifies d-α-tocotrienol, isolated from barley extracts, as an inhibitor of cholesterol biosynthesis. Various human and animal studies have confirmed the impact of pure tocotrienols, isolated from barley, oats, and palm oil, on cholesterol biosynthesis, specifically LDL-cholesterol. In addition, α-tocotrienol, γ- and δ-tocotrienol have been indicated for use in the treatment of hypercholesterolemia, hyperlipidemia, and thromboembolic disorders.

The known naturally occurring tocotrienols have been designated α-, β-, γ, and δ-tocotrienol. Those compounds exhibit varying degrees of hypercholesterolemic activity and have also been used as antithrombotic agents and antioxidants. α-T₃, for example, displays antioxidant activity against lipid peroxidation in rat liver microsomal membranes and against oxidative damage of cytochrome P-450. Despite these activities, tocotrienols have not found wide-spread therapeutic use.

The tocopherol and tocotrienol conjugate compounds of this invention and mixtures thereof also reduce the levels of tumor necrosis factor in response to lipopolysaccharide stimulation, lower arachidonic acid in the tissues and reduce oxygen metabolites in the blood of animals and humans. These results point to an overall reduction in prostaglandins and leukotrienes, both of which are synthesized from arachidonic acid, and a possible reduction in interleukin-1. Accordingly, the compounds of this invention may be employed for a variety of uses. For example, they may be used to prevent endothelial injury, such as ischemic and reperfused myocardium and ulcers. In addition, the inhibition of tumor necrosis factor biosynthesis would also be accompanied by a decrease in inflammation—i.e., through inhibiting the respiratory bursts of neutrophils or through free radical scavenging. Therefore, the compounds of this invention are also useful as antiinflammatory agents for the prevention and treatment of a wide variety of diseases and conditions involving minor, acute and chronic inflammation. These include, but are not limited to, fever, rheumatoid diseases, pain, function laesa, hypertension, and edema.

In addition to their role in inflammatory response, prostaglandins have also been shown to inhibit glucose-induced insulin release, increase glucose concentration, and stimulate glucagon secretion. Consequently, use of the compounds of this invention typically leads to an increased insulin to glucagon ratio. Therefore, the novel tocopherol and tocotrienol conjugate compounds of this invention and mixtures thereof, may be used to improve glucose intolerance in diabetes mellitus. They may also be used to restore acute glucose-induced insulin response in non-insulin-dependent diabetes mellitus.

In addition to the above-stated uses, the tocopherol and tocotrienol conjugate compounds of this invention and mixtures thereof, may also be used to enhance the immune response in animals and humans. These compounds typically reduce the amount of fatty acids in biological tissues. Since fatty acid levels effect the immune system, the compounds of this invention may serve as immunoregulators. They may, for example, be used to increase antibody titers to foreign proteins.

Vitamin E functions as a powerful antioxidant to protect human cells and fatty tissues from free radical damage. Free radicals are extremely dangerous and reactive oxygen compounds that are constantly being produced from a variety of natural sources such as radiation, air pollution, and the breakdown of proteins in the body. Left unchecked, free radicals course throughout the body, rupturing cell membranes, causing massive damage to skin and connective tissues, and damage cellular DNA which gives rise to various cancers and degenerative diseases. Free radical damage also accumulates in the brain, leading to age-related memory impairment.

As antioxidants, tocopherols and tocotrienols protect double bonds in fatty acids from oxidation. Fatty acids are an important part of cell membrane structure. Especially in cells exposed to high concentrations of oxygen, such as red blood cells, white blood cells, lung tissue, and eye tissue, tocopherols and tocotrienols protect cell membranes from oxidative damage. Antioxidation is accomplished in at least two ways. First, by reducing arachidonic acid metabolites, the neutrophils reduce the levels of superoxide production. Second, these compounds scavenge radicals which are already present. Accordingly, they exert a protective effect on the endothelium, lipoproteins, smooth muscle cells, and platelets.

Vitamin E, in combination with other antioxidants, works to quench free radicals and prevent oxidation of polyunsaturated fatty acids that make up cell membranes. By neutralizing free radicals and stabilizing fatty cell membranes, vitamin E helps to prevent cancer, arthritis, immune disorders such as lupus, and premature aging. Working with vitamin A and beta carotene, vitamin E protects the lungs from air pollution. Vitamin E also protects the cells lining blood vessels walls from free radical damage, thus preventing atherosclerosis and cardiovascular disease. By protecting red blood cells from damage, vitamin E also prevents a special form of anemia called hemolytic anemia.

The novel compounds of the present invention have surprisingly been found to effect a variety of biological systems and processes, including aging and longevity, nerve activity, hematopoiesis and maintenance of blood cells, hepatic activity, nephritic activity, heart and cardiovascular function, pulmonary function, muscular function, cartilage, bone, and joint health, gastrointestinal function, reproductive system function, vision, immune function, cell membrane integrity, and pain and inflammation. Further, the compounds of the invention have been found to be useful in preventing or treating diseases or conditions; in treating cancers or obesity; and in reducing the risk of Sudden Infant Death Syndrome. Without being bound to a particular theory or mechanism of action, it appears that covalently linking vitamin E to a vitamin, cofactor, antioxidant compound, or other orthomolecular compound as disclosed herein may enhance the activity of vitamin E and/or the covalently-linked compound in the relevant biochemical pathway.

Although others have recently recognized the significance of orthomolecular methods, Applicant is one of the pioneers in the field. In U.S. Pat. Nos. 5,108,754 (Apr. 28, 1992) and 5,177,208 (Jan. 5, 1993), Applicant obtained patent protection for novel compounds, compositions, and an orthomolecular method for treating sickle cell disease.

Other U.S. patents for orthomolecular methods, as such, include the following: U.S. Pat. No. 4,500,515, for a method for treating alcohol and drug addicts; U.S. Pat. No. 4,876,278, for zinc glycerolate complex and additions for pharmaceutical applications; U.S. Pat. Nos. 4,918,102 and 5,013,752, for prevention and treatment of alcoholism by the use of dietary chromium; U.S. Pat. Nos. 5,070,101 and 5,177,081, for a method and pharmaceutical composition for the treatment of schizophrenia; U.S. Pat. No. 5,230,996, for the use of ascorbate and tranexamic acid solution for organ and blood vessel treatment prior to transplantation; U.S. Pat. No. 5,278,189, for the prevention and treatment of occlusive cardiovascular disease with ascorbate and substances that inhibit the binding of lipoprotein (A); U.S. Pat. No. 5,869,525, for ascorbic acid drugs for intracerebral administration; U.S. Pat. Nos. 5,874,471 and 6,028,107, for the orthomolecular medical use of L-citrulline for vasoprotection, relaxative smooth muscle tone and cell protection; U.S. Pat. No. 5,897,891, for flavorful zinc compositions for oral use incorporating copper; and U.S. Pat. No. 6,039,978, for a dietary food enhancement agent.

Methods of producing stable vitamin E (see U.S. Pat. Nos. 4,191,692, 4,208,334, 4,217,285, 4,252,726, 4,550,182, 4,634,781, 4,639,533, 4,925,960, 5,157,132, 5,457,216, 5,504,220, 5,591,772, 5,663,376, 5,686,632, 5,821,264, 5,886,197, 5,908,940, 5,919,818, and 6,020,505), are known in the art. Applicant has described a process for the synthesis of the α-tocopherol derivative α-(S-adenosylmethionine)-O-tocopherol in U.S. Pat. Nos. 5,108,754 and 5,177,208.

Use of tocopherols or tocotrienols alone is known in the art. Unexpectedly, it has been discovered that the novel covalently-linked compounds of the present invention may enhance the activity of tocopherols or tocotrienols and/or the covalently-linked compound in the relevant biochemical pathway effecting aging and longevity, nerve activity, hematopoiesis and maintenance of blood cells, hepatic activity, nephritic activity, heart and cardiovascular function, pulmonary function, muscular function, cartilage, bone, and joint health, gastrointestinal function, reproductive system function, vision, immune function, cell membrane integrity, and pain and inflammation in an animal. Further, such covalently linked conjugates are effective in preventing or treating diseases or conditions; in treating cancers or obesity; and in reducing the risk of Sudden Infant Death Syndrome.

All of the publications, patents, and other references cited herein are incorporated by reference as though set forth in full.

### SUMMARY OF THE INVENTION

The present invention relates to a novel vitamin E derivative compound of formula I:

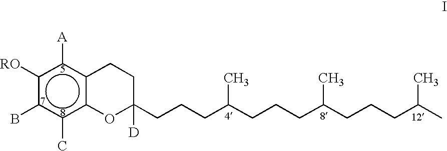

or a pharmaceutically acceptable salt, ester, or solvate, thereof, wherein:

A, B, C, and D are independently hydrogen or methyl;

R is a reaction product derived from a reactant compound selected from the group consisting of (flava-3-ol)ₙ, wherein n is 1-12, α-ketoglutaric acid, α-butyric acid, β-Carotene, 1-methylinosine, 1-methylguanosine, 1-methyladenosine, 1-methylpseudouridine, 1,3-bisphosphoglycerate, 1,3-diphosphoglycerate, 2-methyladenosine, 2-methylthio-N6-isopentenyladenosine, 2-thiouridine, 2-thiocytidine, 2′-O-methylpseudouridine, 2′-O-methyluridine, 2′-O-methylcytidine, 2′-O-methyl-N2,N2-dimethyl-guanosine, 2′-O-methyl-5-methyluridine, 2′-O-methyl-1-methyladenosine, 2′-O-methylguanosine, 2′-O-methyl-N6-methyladenosine, 2′-O-methyladenosine, 2,6-di(tert-butyl)-4-methylphenol, 3-(3-amino-3-carboxypropyl)uridine, 3-carboxy-3-aminopropyl analogues, 3-methylcytidine, 3-methoxy-4-hydroxymandelic acid, 3-phosphoglycerate, 4-thiouridine, 5-carboxymethylaminomethyluridine, 5-methylaminomethyl-2-thiouridine, 5-carboxymethylaminomethyl-2-thiouridine, 5-methylcytidine, 5-methyluridine, 5-(carboxyhydroxymethyl)uridine methyl ester, 5-(carboxyhydroxymethyl)uridine, 5-carbamoylmethyluridine, 5-methyl-2-thiouridine, 5-methoxyuridine, 5-methoxycarbonylmethyl-2-thiouridine, 5-methylaminomethyluridine, 5-methyoxycarbonylmethyluridine, 5-phosphoribose-1-pyrophosphoric acid, 6-gingerol, 7-methylguanosine, acetyl-L-carnitine, acetylcholine, acyl gulycerols, ajoene, alanine, aminocyclopropane-carboxylic acid (ACC), anserine, anthocyanin, apigenin, arachidonic acid, arginine, astaxanthin, beta,D-galactosylqueuosine, beta,D-mannosylqueuosine, betaine, biopterin, biotin coenzymes such as carboxy biotin, biotin, calcium pectate, carbamyl phosphate, carnitine, carnosine, catechin, chlorogenic acid, cholesterol, choline, chondronectin, cis-zeatin, cis-aconitic acid, citric acid, Co-enzyme Q₁₀, cobamide coenzymes such as methylcobalamine or deoxyadenosylcobalamine, creatine, creatinine, cryptoxanthin, cumic acid, cumidine, curcumin, cyanidin chloride, cytochrome B, cytochrome B₅₅₈, cytochrome A, cytochrome C₁, cytochrome A₃, d-limonene, daidzein, diacylglycerol and its derivatives, dicetyl phosphate, diglycerides, dihydrouridine, dihydroxyacetone-phosphate, diphosphatidyl glycerol, diphthamide, dopamine, elastin, ellagic acid, epicatechin gallate, epicatechin, epigallocatechin, epigallocatechin gallate, epinephrine, erythrose-4-phosphate, ether phospholipids, farnesyl, fibronectins, fisetin, flavin coenzymes such as flavin mononucleotide or flavin adenine dinucleotide, flavoxanthine, folic acid, folic or pteridine coenzymes such as tetrahydrofolic acid, formic acid, fructose 1,6-bisphosphate, fructose-6-phosphate, fumaric acid, gallic acid, genistein, geranyl, ginkgolide A, ginkgolide B, ginkgolide C, glucose, glutamic acid, glutamine, glutathione, glyceraldehyde-3-phosphate, glycolipids, glycoproteins, GTP, hesperidin, histamine, histones, HMG Co-A, homoserine lactone, homoserine, indole-3-carbinol, inosinate, inosine, inositol, isocitric acid, isoleucine, kynurenine, L-histidine, L-dopa, laminin, lecithin, leucine, leukotrienes, linatine, lipoic coenzymes such as reduced or oxidized lipoamide, lipoic acid, lupeol, lutein, luteolin, lycopene, lycophyll, lycoxanthine, lysine, lysolecithin, lysophospholipids, malic acid, mandelic acid, melanins, melatonin, metanephrine, methionine, methylated estrogen, methylated lipids, methylated histones, methylated glycolipids, methylated sugars, methylated nucleic acids, methylated proteins, methylated ribosomal proteins, methylated lipids, methylated neurotransmitters, methylated glycoproteins, N-((9-beta-D-ribofuranosylpurine-6-yl)carbamoyl)-threoni ne, N-methylglycine, N-methyl histamine, N-malonyl ACC, N-((9-beta-D-ribofuranosylpurine-6-yl)n-methyl-carbamoyl) threonine, N-((9-beta-D-ribofuranosyl-2-methylthiopurine-2-yl)-carb amoyl)threonine, N-acetyl-neurominic acid, N2-(5-amino-5-carboxypentyl)cytidine, N2-methylguanosine, N2,N2-dimethlyguanosine, N4-acetylcytidine, N6-methyladenosine, N6-isopentenyladenosine, neopterin, nervonic acid, nicotinamide coenzymes such as nicotinamide adenine dinucleotide or nicotinamide adenine dinucleotide phosphate, nicotinic acid, N,N-dimethylglycine, N,N-dimethyltryptamine, norepinephrine, normetanephrine, nucleotide coenzymes such as UDP-glucose, CDP-choline, CDP-diacylglycerol, CMP-sialic acid, and other nucleotide derivatives of carbohydrates, alcohols, amino acids, lipids, or inorganic compounds, Omega-3 fatty acids, ornithine, oxaloacetic acid, p-coumaric acid, palmitic acid, pantothenic coenzymes such as pantothenic CoA, pantothenic dephospho-CoA, or 4-phosphopantothenate, pantothenic acid, para-aminobenzoic acid (PABA), pectin, phenylalanine, phosphatidyl ethanolamine, phosphatidyl choline, phosphocreatine, phosphoenol pyruvate, phosphonolipids, phytic acid, phytochlorin, phytol, picolinic acid, plasmalogens, plastoquinone, proanthocyanin, pseudouridine, pyridoxine coenzymes such as pyridoxal-phosphate or pyridoxamine-phosphate, pyruvate, pyruvate, quercetin, queuine, queuosine, quinolinic acid, ribose-5-phosphate, ribosomal proteins, ribulose-1,5-biphosphate, rutin, S-allymercaptocysteine, sarcosine, sedoheptulose-1,7-bisphosphate, sedoheptulose-7-phosphate, serotonin, sesamin, silybin, soy isoflavonoids, sphingolipids, sphingomyelin, sphingosine, steraylamine, succinic acid, sugars such as lactose, glucose, or mannose, sulforaphone, sulphorane, taurine, taxicatin, taxicin I, taxicin II, taxifolin, taxine A, taxodione, testosterone, tetrahydrobiopterin and its derivatives, tetrahydrofolate, thiamine coenzymes such as thiamine monophosphate, thiamine diphosphate, or thiamine triphosphate, threonine, trimethylysine, tryptamine, tryptophane, tumeric, tyrosine, uridine-5-oxyacetic acid, uridine-5-oxoacetic acid methyl ester, vaccenic acid, valine, vanillic acid, vitamin A, vitamin B₁ (Thiamine), vitamin B₂ (Riboflavin), vitamin B₆ (Pyrodoxine), vitamin B₁₂ (Cyanocobalamine), vitamin C (esterified or non-esterified Ascorbic Acid), vitamin D (Ergocalciferol), wybutosine, wybutoxosine, wye base, xanthophyll, xanthoxylin, xylulose-5-phosphate, and zeaxanthin;

R is a straight or branched C₁-C₃₀ alkyl, or C₂-C₃₀ straight or branched alkenyl or alkynyl optionally substituted with 1 to 12 substituents selected from the group consisting of hydroxy, carboxy, amino, halo, nitro, sulfhydryl, and J, wherein J is phenyl or a 5-7 membered heterocyclic ring which has one or more O, N, or S as the heteroatom(s), and J is optionally substituted with 1 to 5 substituents selected from the group consisting of hydroxy, carboxy, amino, halo, nitro, sulfhydryl, methyl, straight or branched C₂-C₁₀ alkyl, alkenyl, or alkynyl, methoxy, C₂-C₈ straight or branched alkoxy, and —OC(O)R₂ where R₂ is trifluoromethyl, methyl, straight or branched C₁-C₁₀ alkyl, or straight or branched C₂-C₁₀ alkenyl or alkynyl, wherein said hydroxy, carboxy, amino, halo, nitro, sulfhydryl, C₁-C₁₀ alkyl, C₂-C₁₀ alkenyl or alkynyl, methoxy, C₂-C₈ straight or branched alkoxy, and —OC(O)R₂ substituents are optionally substituted; and wherein one or more carbon atom(s) of said alkyl, alkenyl, alkynyl, or alkoxy, is optionally replaced with nitrogen, oxygen, or sulfur; or

R is phenyl optionally substituted with 1 to 5substituents selected from the group consisting of hydroxy, carboxy, amino, halo, nitro, sulfhydryl, trifluoromethyl, methyl, C₁-C₈ straight or branched alkyl, C₂-C₈ straight or branched alkenyl or alkynyl, methoxy, and C₂-C₈ straight or branched alkoxy, wherein said hydroxy, carboxy, amino, sulfhydryl, halo, nitro, C₁-C₈ straight or branched alkyl, C₂-C₈ straight or branched alkenyl or alkynyl, methoxy, and C₂-C₈ straight or branched alkoxy substituents are optionally substituted; and wherein one or more carbon atom(s) of said alkyl, alkenyl, alkynyl, or alkoxy, is optionally replaced with nitrogen, oxygen, or sulfur;

and wherein the stereochemistry at each of the 2, 4′, and 8′ positions is R or S.

The present invention also relates to a novel vitamin E derivative compound of formula II

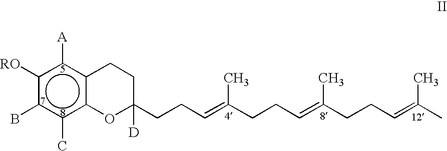

or a pharmaceutically acceptable salt, ester, or solvate thereof, wherein:

A, B, C, and D are independently hydrogen or methyl;

R is a reaction product derived from a reactant compound selected from the group consisting of (flava-3-ol)ₙ, wherein n is 1-12, α-ketoglutaric acid, α-butyric acid, β-Carotene, 1-methylinosine, 1-methylguanosine, 1-methyladenosine, 1-methylpseudouridine, 1,3-bisphosphoglycerate, 1,3-diphosphoglycerate, 2-methyladenosine, 2-methylthio-N6-isopentenyladenosine, 2-thiouridine, 2-thiocytidine, 2′-O-methylpseudouridine, 2′-O-methyluridine, 2′-O-methylcytidine, 2′-O-methyl-N2,N2-dimethyl-guanosine, 2′-O-methyl-5-methyluridine, 2′-O-methyl-1-methyladenosine, 2′-O-methylguanosine, 2′-O-methyl-N6-methyladenosine, 2′-O-methyladenosine, 2,6-di(tert-butyl)-4-methylphenol, 3-(3-amino-3-carboxypropyl)uridine, 3-carboxy-3-aminopropyl analogues, 3-methylcytidine, 3-methoxy-4-hydroxymandelic acid, 3-phosphoglycerate, 4-thiouridine, 5-carboxymethylaminomethyluridine, 5-methylaminomethyl-2-thiouridine, 5-carboxymethylaminomethyl-2-thiouridine, 5-methylcytidine, 5-methyluridine; 5-(carboxyhydroxymethyl)uridine methyl ester, 5-(carboxyhydroxymethyl)uridine, 5-carbamoylmethyluridine, 5-methyl-2-thiouridine, 5-methoxyuridine, 5-methoxycarbonylmethyl-2-thiouridine, 5-methylaminomethyluridine, 5-methyoxycarbonylmethyluridine, 5-phosphoribose-1-pyrophosphoric acid, 6-gingerol, 7-methylguanosine, acetyl-L-carnitine, acetylcholine, acyl gulycerols, ajoene, alanine, aminocyclopropane-carboxylic acid (ACC), anserine, anthocyanin, apigenin, arachidonic acid, arginine, astaxanthin, beta,D-galactosylqueuosine, beta,D-mannosylqueuosine, betaine, biopterin, biotin coenzymes such as carboxy biotin, biotin, calcium pectate, carbamyl phosphate, carnitine, carnosine, catechin, chlorogenic acid, cholesterol, choline, chondronectin, cis-zeatin, cis-aconitic acid, citric acid, Co-enzyme Q₁₀, cobamide coenzymes such as methylcobalamine or deoxyadenosylcobalamine, creatine, creatinine, cryptoxanthin, cumic acid, cumidine, curcumin, cyanidin chloride, cytochrome B, cytochrome B₅₅₈, cytochrome A, cytochrome C₁, cytochrome A₃, d-limonene, daidzein, diacylglycerol and its derivatives, dicetyl phosphate, diglycerides, dihydrouridine, dihydroxyacetone-phosphate, diphosphatidyl glycerol, diphthamide, dopamine, elastin, ellagic acid, epicatechin gallate, epicatechin, epigallocatechin, epigallocatechin gallate, epinephrine, erythrose-4-phosphate, ether phospholipids, farnesyl, fibronectins, fisetin, flavin coenzymes such as flavin mononucleotide or flavin adenine dinucleotide, flavoxanthine, folic acid, folic or pteridine coenzymes such as tetrahydrofolic acid, formic acid, fructose 1,6-bisphosphate, fructose-6-phosphate, fumaric acid, gallic acid, genistein, geranyl, ginkgolide A, ginkgolide B, ginkgolide C, glucose, glutamic acid, glutamine, glutathione, glyceraldehyde-3-phosphate, glycolipids, glycoproteins, GTP, hesperidin, histamine, histones, HMG Co-A, homoserine lactone, homoserine, indole-3-carbinol, inosinate, inosine, inositol, isocitric acid, isoleucine, kynurenine, L-histidine, L-dopa, laminin, lecithin, leucine, leukotrienes, linatine, lipoic coenzymes such as reduced or oxidized lipoamide, lipoic acid, lupeol, lutein, luteolin, lycopene, lycophyll, lycoxanthine, lysine, lysolecithin, lysophospholipids, malic acid, mandelic acid, melanins, melatonin, metanephrine, methionine, methylated estrogen, methylated lipids, methylated histones, methylated glycolipids, methylated sugars, methylated nucleic acids, methylated proteins, methylated ribosomal proteins, methylated lipids, methylated neurotransmitters, methylated glycoproteins, N-((9-beta-D-ribofuranosylpurine-6-yl)carbamoyl)-threoni ne, N-methylglycine, N-methyl histamine, N-malonyl ACC, N-((9-beta-D-ribofuranosylpurine-6-yl)n-methyl-carbamoyl) threonine, N-((9-beta-D-ribofuranosyl-2-methylthiopurine-2-yl)-carb amoyl)threonine, N-acetyl-neurominic acid, N2-(5-amino-5-carboxypentyl)cytidine, N2-methylguanosine, N2,N2-dimethlyguanosine, N4-acetylcytidine, N6-methyladenosine, N6-isopentenyladenosine, neopterin, nervonic acid, nicotinamide coenzymes such as nicotinamide adenine dinucleotide or nicotinamide adenine dinucleotide phosphate, nicotinic acid, N,N-dimethylglycine, N,N-dimethyltryptamine, norepinephrine, normetanephrine, nucleotide coenzymes such as UDP-glucose, CDP-choline, CDP-diacylglycerol, CMP-sialic acid, and other nucleotide derivatives of carbohydrates, alcohols, amino acids, lipids, or inorganic compounds, Omega-3 fatty acids, ornithine, oxaloacetic acid, p-coumaric acid, palmitic acid, pantothenic coenzymes such as pantothenic CoA, pantothenic dephospho-CoA, or 4-phosphopantothenate, pantothenic acid, para-aminobenzoic acid (PABA), pectin, phenylalanine, phosphatidyl ethanolamine, phosphatidyl choline, phosphocreatine, phosphoenol pyruvate, phosphonolipids, phytic acid, phytochlorin, phytol, picolinic acid, plasmalogens, plastoquinone, proanthocyanin, pseudouridine, pyridoxine coenzymes such as pyridoxal-phosphate or pyridoxamine-phosphate, pyruvate, pyruvate, quercetin, queuine, queuosine, quinolinic acid, ribose-5-phosphate, ribosomal proteins, ribulose-1,5-biphosphate, rutin, S-allymercaptocysteine, sarcosine, sedoheptulose-1,7-bisphosphate, sedoheptulose-7-phosphate, serotonin, sesamin, silybin, soy isoflavonoids, sphingolipids, sphingomyelin, sphingosine, steraylamine, succinic acid, sugars such as lactose, glucose, or mannose, sulforaphone, sulphorane, taurine, taxicatin, taxicin I, taxicin II, taxifolin, taxine A, taxodione, testosterone, tetrahydrobiopterin and its derivatives, tetrahydrofolate, thiamine coenzymes such as thiamine monophosphate, thiamine diphosphate, or thiamine triphosphate, threonine, trimethylysine, tryptamine, tryptophane, tumeric, tyrosine, uridine-5-oxyacetic acid, uridine-5-oxoacetic acid methyl ester, vaccenic acid, valine, vanillic acid, vitamin A, vitamin B₁, (Thiamine), vitamin B₂ (Riboflavin), vitamin B₆ (Pyrodoxine), vitamin B₁₂ (Cyanocobalamine), vitamin C (esterified or non-esterified Ascorbic Acid), vitamin D (Ergocalciferol), wybutosine, wybutoxosine, wye base, xanthophyll, xanthoxylin, xylulose-5-phosphate, and zeaxanthin;

R is a straight or branched C₁-C₃₀ alkyl, or C₂-C₃₀ straight or branched alkenyl or alkynyl optionally substituted with 1 to 12 substituents selected from the group consisting of hydroxy, carboxy, amino, halo, nitro, sulfhydryl, and J, wherein J is phenyl or a 5-7 membered heterocyclic ring which has one or more O, N, or S as the heteroatom(s), and J is optionally substituted with 1 to 5 substituents selected from the group consisting of hydroxy, carboxy, amino, halo, nitro, sulfhydryl, methyl, straight or branched C₂-C₁₀ alkyl, alkenyl, or alkynyl, methoxy, C₂-C₈ straight or branched alkoxy, and —OC(O)R₂ where R₂ is trifluoromethyl, methyl, straight or branched C₁-C₁₀ alkyl, or straight or branched C₂-C₁₀ alkenyl or alkynyl, wherein said hydroxy, carboxy, amino, halo, nitro, sulfhydryl, C₁-C₁₀ alkyl, C₂-C₁₀ alkenyl or alkynyl, methoxy, C₂-C₈ straight or branched alkoxy, and —OC(O)R₂ substituents are optionally substituted; and wherein one or more carbon atom(s) of said alkyl, alkenyl, alkynyl, or alkoxy, is optionally replaced with nitrogen, oxygen, or sulfur; or

R is phenyl optionally substituted with 1 to 5 substituents selected from the group consisting of hydroxy, carboxy, amino, halo, nitro, sulfhydryl, trifluoromethyl, methyl, C₁-C₈ straight or branched alkyl, C₂-C₈ straight or branched alkenyl or alkynyl, methoxy, and C₂-C₈ straight or branched alkoxy, wherein said hydroxy, carboxy, amino, sulfhydryl, halo, nitro, C₁-C₈ straight or branched alkyl, C₂-C₈ straight or branched alkenyl or alkynyl, methoxy, and C₂-C₈ straight or branched alkoxy substituents are optionally substituted; and wherein one or more carbon atom(s) of said alkyl, alkenyl, alkynyl, or alkoxy, is optionally replaced with nitrogen, oxygen, or sulfur;

and wherein the stereochemistry at each of the 2, 4′, and 8′ positions is R or S.

The present invention further relates to a method for preventing or treating diseases or conditions; effecting aging and longevity, nerve activity, hematopoiesis and maintenance of blood cells, hepatic activity, nephritic activity, heart and cardiovascular function, pulmonary function, muscular function, cartilage, bone, and joint health, gastrointestinal function, reproductive system function, vision, immune function, cell membrane integrity, and pain and inflammation; preventing or treating diseases or conditions; treating cancers or obesity; and in reducing the risk of Sudden Infant Death Syndrome in an animal, which comprises administering to said animal an effective amount of a vitamin E derivative compound of formula I or formula II.

Finally, the present invention relates to a pharmaceutical composition comprising:

(i) an effective amount of a vitamin E derivative compound of formula I or II; and

(ii) a pharmaceutically acceptable carrier.

### DETAILED DESCRIPTION OF THE INVENTION

### Definitions

“Effecting” refers to the process of producing an effect on biological activity, function, health, or condition of an organism in which such biological activity, function, health, or condition is maintained, enhanced, diminished, or treated in a manner which is consistent with the general health and well-being of the organism.

“Enhancing” the biological activity, function, health, or condition of an organism refers to the process of augmenting, fortifying, strengthening, or improving.

“Isomers” refer to different compounds that have the same molecular formula. “Stereoisomers” are isomers that differ only in the way the atoms are arranged in space. “Enantiomers” are a pair of stereoisomers that are non-superimposable mirror images of each other. “Diastereoisomers” are stereoisomers which are not mirror images of each other. “Racemic mixture” means a mixture containing equal parts of individual enantiomers. “Non-racemic mixture” is a mixture containing unequal parts of individual enantiomers or stereoisomers.

“Pharmaceutically acceptable salt, ester, or solvate” refers to a salt, ester, or solvate of a subject compound which possesses the desired pharmacological activity and which is neither biologically nor otherwise undesirable. A salt, ester, or solvate can be formed with inorganic acids such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, gluconate, glycerophosphate, hemisulfate, heptanoate, hexanoate, hydrochloride, hydrobromide, hydroiodide, 2-hydroxyethanesulfonate, lactate, maleate, methanesulfonate, naphthylate, 2-naphthalenesulfonate, nicotinate, oxalate, sulfate, thiocyanate, tosylate, and undecanoate. Examples of base salts, esters, or solvates include ammonium salts; alkali metal salts, such as sodium and potassium salts; alkaline earth metal salts, such as calcium and magnesium salts; salts with organic bases, such as dicyclohexylamine salts; N-methyl-D-glucamine; and salts with amino acids, such as arginine, lysine, and so forth. Also, the basic nitrogen-containing groups can be quarternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; aralkyl halides, such as benzyl and phenethyl bromides; and others. Water or oil-soluble or dispersible products are thereby obtained.

It is to be understood that, in its most common form, a “reactant compound” within the scope of the present invention may or may not have the reactive moiety(ies) necessary to produce a compound of the present invention. It is intended that such compound(s) will be derivatized to add one or more reactive moiety(ies) by means known to one of ordinary skill in the art. By way of example and not limitation, appropriate derivatives may be produced by hydration, halogenation, carboxylation, amination, nitration, and sulfonation.

“Reaction product” refers to that part of a reactant compound remaining after the chemical reaction producing a covalently-linked compound of the present invention. Such chemical reactions include substitution, elimination, addition, oxidation, and reduction reactions, and involve reactive moieties such as multiple bonds; oxygen and hydroxyl; nitrogen, nitro, amide, and amine; sulfur, sulfhydryl, and sulpho; and other common groups known to one of ordinary skill in the art.

“Vitamin E” refers to a compound of formula I:

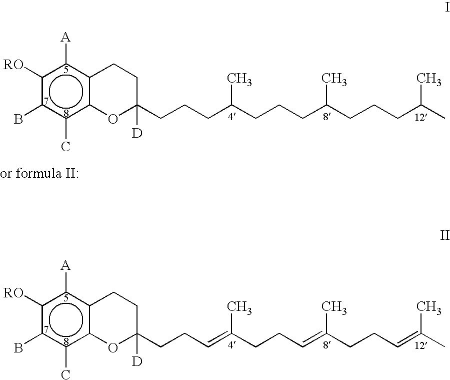

or a pharmaceutically acceptable salt, ester, or solvate, thereof, wherein:

A, B, C, and D are independently hydrogen or methyl;

R is a reaction product derived from a reactant compound selected from the group consisting of (flava-3-ol)ₙ, wherein n is 1-12, α-ketoglutaric acid, α-butyric acid, β-Carotene, 1-methylinosine, 1-methylguanosine, 1-methyladenosine, 1-methylpseudouridine, 1,3-bisphosphoglycerate, 1,3-diphosphoglycerate, 2-methyladenosine, 2-methylthio-N6-isopentenyladenosine, 2-thiouridine, 2-thiocytidine, 2′-O-methylpseudouridine, 2′-O-methyluridine, 2′-O-methylcytidine, 2′-O-methyl-N2,N2-dimethyl-guanosine, 2′-O-methyl-5-methyluridine, 2′-O-methyl-1-methyladenosine, 2′-O-methylguanosine, 2′-O-methyl-N6-methyladenosine, 2′-O-methyladenosine, 2,6-di(tert-butyl)-4-methylphenol, 3-(3-amino-3-carboxypropyl)uridine, 3-carboxy-3-aminopropyl analogues, 3-methylcytidine, 3-methoxy-4-hydroxymandelic acid, 3-phosphoglycerate, 4-thiouridine, 5-carboxymethylaminomethyluridine, 5-methylaminomethyl-2-thiouridine, 5-carboxymethylaminomethyl-2-thiouridine, 5-methylcytidine, 5-methyluridine, 5-(carboxyhydroxymethyl)uridine methyl ester, 5-(carboxyhydroxymethyl)uridine, 5-carbamoylmethyluridine, 5-methyl-2-thiouridine, 5-methoxyuridine, 5-methoxycarbonylmethyl-2-thiouridine, 5-methylaminomethyluridine, 5-methyoxycarbonylmethyluridine, 5-phosphoribose-1-pyrophosphoric acid, 6-gingerol, 7-methylguanosine, acetyl-L-carnitine, acetylcholine, acyl gulycerols, ajoene, alanine, aminocyclopropane-carboxylic acid (ACC), anserine, anthocyanin, apigenin, arachidonic acid, arginine, astaxanthin, beta,D-galactosylqueuosine, beta,D-mannosylqueuosine, betaine, biopterin, biotin coenzymes such as carboxy biotin, biotin, calcium pectate, carbamyl phosphate, carnitine, carnosine, catechin, chlorogenic acid, cholesterol, choline, chondronectin, cis-zeatin, cis-aconitic acid, citric acid, Co-enzyme Q₁₀, cobamide coenzymes such as methylcobalamine or deoxyadenosylcobalamine, creatine, creatinine, cryptoxanthin, cumic acid, cumidine, curcumin, cyanidin chloride, cytochrome B, cytochrome B₅₅₈, cytochrome A, cytochrome C₁, cytochrome A₃, d-limonene, daidzein, diacylglycerol and its derivatives, dicetyl phosphate, diglycerides, dihydrouridine, dihydroxyacetone-phosphate, diphosphatidyl glycerol, diphthamide, dopamine, elastin, ellagic acid, epicatechin gallate, epicatechin, epigallocatechin, epigallocatechin gallate, epinephrine, erythrose-4-phosphate, ether phospholipids, farnesyl, fibronectins, fisetin, flavin coenzymes such as flavin mononucleotide or flavin adenine dinucleotide, flavoxanthine, folic acid, folic or pteridine coenzymes such as tetrahydrofolic acid, formic acid, fructose 1,6-bisphosphate, fructose-6-phosphate, fumaric acid, gallic acid, genistein, geranyl, ginkgolide A, ginkgolide B, ginkgolide C, glucose, glutamic acid, glutamine, glutathione, glyceraldehyde-3-phosphate, glycolipids, glycoproteins, GTP, hesperidin, histamine, histones, HMG Co-A, homoserine lactone, homoserine, indole-3-carbinol, inosinate, inosine, inositol, isocitric acid, isoleucine, kynurenine, L-histidine, L-dopa, laminin, lecithin, leucine, leukotrienes, linatine, lipoic coenzymes such as reduced or oxidized lipoamide, lipoic acid, lupeol, lutein, luteolin, lycopene, lycophyll, lycoxanthine, lysine, lysolecithin, lysophospholipids, malic acid, mandelic acid, melanins, melatonin, metanephrine, methionine, methylated estrogen, methylated lipids, methylated histones, methylated glycolipids, methylated sugars, methylated nucleic acids, methylated proteins, methylated ribosomal proteins, methylated lipids, methylated neurotransmitters, methylated glycoproteins, N-((9-beta-D-ribofuranosylpurine-6-yl)carbamoyl)-threoni ne, N-methylglycine, N-methyl histamine, N-malonyl ACC, N-((9-beta-D-ribofuranosylpurine-6-yl)n-methyl-carbamoyl) threonine, N-((9-beta-D-ribofuranosyl-2-methylthiopurine-2-yl)-carb amoyl)threonine, N-acetyl-neurominic acid, N2-(5-amino-5-carboxypentyl)cytidine, N2-methylguanosine, N2,N2-dimethlyguanosine, N4-acetylcytidine, N6-methyladenosine, N6-isopentenyladenosine, neopterin, nervonic acid, nicotinamide coenzymes such as nicotinamide adenine dinucleotide or nicotinamide adenine dinucleotide phosphate, nicotinic acid, N,N-dimethylglycine, N,N-dimethyltryptamine, norepinephrine, normetanephrine, nucleotide coenzymes such as UDP-glucose, CDP-choline, CDP-diacylglycerol, CMP-sialic acid, and other nucleotide derivatives of carbohydrates, alcohols, amino acids, lipids, or inorganic compounds, Omega-3 fatty acids, ornithine, oxaloacetic acid, p-coumaric acid, palmitic acid, pantothenic coenzymes such as pantothenic CoA, pantothenic dephospho-CoA, or 4-phosphopantothenate, pantothenic acid, para-aminobenzoic acid (PABA), pectin, phenylalanine, phosphatidyl ethanolamine, phosphatidyl choline, phosphocreatine, phosphoenol pyruvate, phosphonolipids, phytic acid, phytochlorin, phytol, picolinic acid, plasmalogens, plastoquinone, proanthocyanin, pseudouridine, pyridoxine coenzymes such as pyridoxal-phosphate or pyridoxamine-phosphate, pyruvate, pyruvate, quercetin, queuine, queuosine, quinolinic acid, ribose-5-phosphate, ribosomal proteins, ribulose-1,5-biphosphate, rutin, S-allymercaptocysteine, sarcosine, sedoheptulose-1,7-bisphosphate, sedoheptulose-7-phosphate, serotonin, sesamin, silybin, soy isoflavonoids, sphingolipids, sphingomyelin, sphingosine, steraylamine, succinic acid, sugars such as lactose, glucose, or mannose, sulforaphone, sulphorane, taurine, taxicatin, taxicin I, taxicin II, taxifolin, taxine A, taxodione, testosterone, tetrahydrobiopterin and its derivatives, tetrahydrofolate, thiamine coenzymes such as thiamine monophosphate, thiamine diphosphate, or thiamine triphosphate, threonine, trimethylysine, tryptamine, tryptophane, tumeric, tyrosine, uridine-5-oxyacetic acid, uridine-5-oxoacetic acid methyl ester, vaccenic acid, valine, vanillic acid, vitamin A, vitamin B₁ (Thiamine), vitamin B₂ (Riboflavin), vitamin B₆ (Pyrodoxine), vitamin B₁₂ (Cyanocobalamine), vitamin C (esterified or non-esterified Ascorbic Acid), vitamin D (Ergocalciferol), wybutosine, wybutoxosine, wye base, xanthophyll, xanthoxylin, xylulose-5-phosphate, and zeaxanthin;

R is a straight or branched C₁-C₃₀ alkyl, or C₂-C₃₀ straight or branched alkenyl or alkynyl optionally substituted with 1 to 12 substituents selected from the group consisting of hydroxy, carboxy, amino, halo, nitro, sulfhydryl, and J, wherein J is phenyl or a 5-7 membered heterocyclic ring which has one or more O, N, or S as the heteroatom(s), and J is optionally substituted with 1 to 5 substituents selected from the group consisting of hydroxy, carboxy, amino, halo, nitro, sulfhydryl, methyl, straight or branched C₂-C₁₀ alkyl, alkenyl, or alkynyl, methoxy, C₂-C₈ straight or branched alkoxy, and —OC(O)R₂ where R₂ is trifluoromethyl, methyl, straight or branched C₁-C₁₀ alkyl, or straight or branched C₂-C₁₀ alkenyl or alkynyl, wherein said hydroxy, carboxy, amino, halo, nitro, sulfhydryl, C₁-C₁₀ alkyl, C₂-C₁₀ alkenyl or alkynyl, methoxy, C₂-C₈ straight or branched alkoxy, and —OC(O)R₂ substituents are optionally substituted; and wherein one or more carbon atom(s) of said alkyl, alkenyl, alkynyl, or alkoxy, is optionally replaced with nitrogen, oxygen, or sulfur; or

R is phenyl optionally substituted with 1 to 5 substituents selected from the group consisting of hydroxy, carboxy, amino, halo, nitro, sulfhydryl, trifluoromethyl, methyl, C₁-C₈ straight or branched alkyl, C₂-C₈ straight or branched alkenyl or alkynyl, methoxy, and C₂-C₈ straight or branched alkoxy, wherein said hydroxy, carboxy, amino, sulfhydryl, halo, nitro, C₁-C₈ straight or branched alkyl, C₂-C₈ straight or branched alkenyl or alkynyl, methoxy, and C₂-C₈ straight or branched alkoxy substituents are optionally substituted; and wherein one or more carbon atom(s) of said alkyl, alkenyl, alkynyl, or alkoxy, is optionally replaced with nitrogen, oxygen, or sulfur;

and wherein the stereochemistry at each of the 2, 4′, and 8′ positions is R or S.

“Treating” refers to:

(i) preventing a disease and/or condition from occurring in a subject which may be predisposed to the disease and/or condition but has not yet been diagnosed as having it;

(ii) inhibiting the disease and/or condition, i.e., arresting its development; or

(iii) relieving the disease and/or condition, i.e., causing regression of the disease and/or condition.

### Orthomolecular Vitamin E Derivatives

The present invention relates to novel vitamin E derivative compounds of Formula I.

### Formula I

The vitamin E derivative may be a compound of formula I:

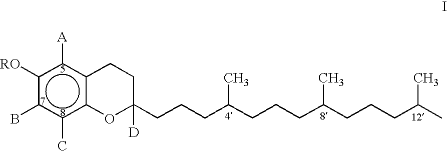

or a pharmaceutically acceptable salt, ester, or solvate, thereof, wherein:

A, B, C, and D are independently hydrogen or methyl;

R is a reaction product derived from a reactant compound selected from the group consisting of (flava-3-ol)ₙ, wherein n is 1-12, α-ketoglutaric acid, α-butyric acid, β-Carotene, 1-methylinosine, 1-methylguanosine, 1-methyladenosine, 1-methylpseudouridine, 1,3-bisphosphoglycerate, 1,3-diphosphoglycerate, 2-methyladenosine, 2-methylthio-N6-isopentenyladenosine, 2-thiouridine, 2-thiocytidine, 2′-O-methylpseudouridine, 2′-O-methyluridine, 2′-O-methylcytidine, 2′-methyl-N2,N2-dimethyl-guanosine, 2′-O-methyl-5-methyluridine, 2′-O-methyl-1-methyladenosine, 2-O-methylguanosine, 2′-O-methyl-N6-methyladenosine, 2′-O-methyladenosine, 2,6-di(tert-butyl)-4-methylphenol, 3-(3-amino-3-carboxypropyl)uridine, 3-carboxy-3-aminopropyl analogues, 3-methylcytidine, 3-methoxy-4-hydroxymandelic acid, 3-phosphoglycerate, 4-thiouridine, 5-carboxymethylaminomethyluridine, 5-methylaminomethyl-2-thiouridine, 5-carboxymethylaminomethyl-2-thiouridine, 5-methylcytidine, 5-methyluridine, 5-(carboxyhydroxymethyl)uridine methyl ester, 5-(carboxyhydroxymethyl)uridine, 5-carbamoylmethyluridine, 5-methyl-2-thiouridine, 5-methoxyuridine, 5-methoxycarbonylmethyl-2-thiouridine, 5-methylaminomethyluridine, 5-methyoxycarbonylmethyluridine, 5-phosphoribose-1-pyrophosphoric acid, 6-gingerol, 7-methylguanosine, acetyl-L-carnitine, acetylcholine, acyl gulycerols, ajoene, alanine, aminocyclopropane-carboxylic acid (ACC), anserine, anthocyanin, apigenin, arachidonic acid, arginine, astaxanthin, beta,D-galactosylqueuosine, beta,D-mannosylqueuosine, betaine, biopterin, biotin coenzymes such as carboxy biotin, biotin, calcium pectate, carbamyl phosphate, carnitine, carnosine, catechin, chlorogenic acid, cholesterol, choline, chondronectin, cis-zeatin, cis-aconitic acid, citric acid, Co-enzyme Q₁₀, cobamide coenzymes such as methylcobalamine or deoxyadenosylcobalamine, creatine, creatinine, cryptoxanthin, cumic acid, cumidine, curcumin, cyanidin chloride, cytochrome B, cytochrome B₅₅₈, cytochrome A, cytochrome C₁, cytochrome A₃, d-limonene, daidzein, diacylglycerol and its derivatives, dicetyl phosphate, diglycerides, dihydrouridine, dihydroxyacetone-phosphate, diphosphatidyl glycerol, diphthamide, dopamine, elastin, ellagic acid, epicatechin gallate, epicatechin, epigallocatechin, epigallocatechin gallate, epinephrine, erythrose-4-phosphate, ether phospholipids, farnesyl, fibronectins, fisetin, flavin coenzymes such as flavin mononucleotide or flavin adenine dinucleotide, flavoxanthine, folic acid, folic or pteridine coenzymes such as tetrahydrofolic acid, formic acid, fructose 1,6-bisphosphate, fructose-6-phosphate, fumaric acid, gallic acid, genistein, geranyl, ginkgolide A, ginkgolide B, ginkgolide C, glucose, glutamic acid, glutamine, glutathione, glyceraldehyde-3-phosphate, glycolipids, glycoproteins, GTP, hesperidin, histamine, histones, HMG Co-A, homoserine lactone, homoserine, indole-3-carbinol, inosinate, inosine, inositol, isocitric acid, isoleucine, kynurenine, L-histidine, L-dopa, laminin, lecithin, leucine, leukotrienes, linatine, lipoic coenzymes such as reduced or oxidized lipoamide, lipoic acid, lupeol, lutein, luteolin, lycopene, lycophyll, lycoxanthine, lysine, lysolecithin, lysophospholipids, malic acid, mandelic acid, melanins, melatonin, metanephrine, methionine, methylated estrogen, methylated lipids, methylated histones, methylated glycolipids, methylated sugars, methylated nucleic acids, methylated proteins, methylated ribosomal proteins, methylated lipids, methylated neurotransmitters, methylated glycoproteins, N-((9-beta-D-ribofuranosylpurine-6-yl)carbamoyl)-threoni ne, N-methylglycine, N-methyl histamine, N-malonyl ACC, N-((9-beta-D-ribofuranosylpurine-6-yl)n-methyl-carbamoyl) threonine, N-((9-beta-D-ribofuranosyl-2-methylthiopurine-2-yl)-carb amoyl)threonine, N-acetyl-neurominic acid, N2-(5-amino-5-carboxypentyl)cytidine, N2-methylguanosine, N2,N2-dimethlyguanosine, N4-acetylcytidine, N6-methyladenosine, N6-isopentenyladenosine, neopterin, nervonic acid, nicotinamide coenzymes such as nicotinamide adenine dinucleotide or nicotinamide adenine dinucleotide phosphate, nicotinic acid, N,N-dimethylglycine, N,N-dimethyltryptamine, norepinephrine, normetanephrine, nucleotide coenzymes such as UDP-glucose, CDP-choline, CDP-diacylglycerol, CMP-sialic acid, and other nucleotide derivatives of carbohydrates, alcohols, amino acids, lipids, or inorganic compounds, Omega-3 fatty acids, ornithine, oxaloacetic acid, p-coumaric acid, palmitic acid, pantothenic coenzymes such as pantothenic CoA, pantothenic dephospho-CoA, or 4-phosphopantothenate, pantothenic acid, para-aminobenzoic acid (PABA), pectin, phenylalanine, phosphatidyl ethanolamine, phosphatidyl choline, phosphocreatine, phosphoenol pyruvate, phosphonolipids, phytic acid, phytochlorin, phytol, picolinic acid, plasmalogens, plastoquinone, proanthocyanin, pseudouridine, pyridoxine coenzymes such as pyridoxal-phosphate or pyridoxamine-phosphate, pyruvate, pyruvate, quercetin, queuine, queuosine, quinolinic acid, ribose-5-phosphate, ribosomal proteins, ribulose-1,5-biphosphate, rutin, S-allymercaptocysteine, sarcosine, sedoheptulose-1,7-bisphosphate, sedoheptulose-7-phosphate, serotonin, sesamin, silybin, soy isoflavonoids, sphingolipids, sphingomyelin, sphingosine, steraylamine, succinic acid, sugars such as lactose, glucose, or mannose, sulforaphone, sulphorane, taurine, taxicatin, taxicin I, taxicin II, taxifolin, taxine A, taxodione, testosterone, tetrahydrobiopterin and its derivatives, tetrahydrofolate, thiamine coenzymes such as thiamine monophosphate, thiamine diphosphate, or thiamine triphosphate, threonine, trimethylysine, tryptamine, tryptophane, tumeric, tyrosine, uridine-5-oxyacetic acid, uridine-5-oxcacetic acid methyl ester, vaccenic acid, valine, vanillic acid, vitamin A, vitamin B₁, (Thiamine), vitamin B₂ (Riboflavin), vitamin B₆ (Pyrodoxine), vitamin B₁₂ (Cyanocobalamine), vitamin C (esterified or non-esterified Ascorbic Acid), vitamin D (Ergocalciferol), wybutosine, wybutoxosine, wye base, xanthophyll, xanthoxylin, xylulose-5-phosphate, and zeaxanthin;

R is a straight or branched C₁-C₃₀ alkyl, or C₂-C₃₀ straight or branched alkenyl or alkynyl optionally substituted with 1 to 12 substituents selected from the group consisting of hydroxy, carboxy, amino, halo, nitro, sulfhydryl, and J, wherein J is phenyl or a 5-7 membered heterocyclic ring which has one or more O, N, or S as the heteroatom(s), and J is optionally substituted with 1 to 5 substituents selected from the group consisting of hydroxy, carboxy, amino, halo, nitro, sulfhydryl, methyl, straight or branched C₂-C₁₀ alkyl, alkenyl, or alkynyl, methoxy, C₂-C₈ straight or branched alkoxy, and —OC(O)R₂ where R₂ is trifluoromethyl, methyl, straight or branched C₁-C₁₀ alkyl, or straight or branched C₂-C₁₀ alkenyl or alkynyl, wherein said hydroxy, carboxy, amino, halo, nitro, sulfhydryl, C₁-C₁₀ alkyl, C₂-C₁₀ alkenyl or alkynyl, methoxy, C₂-C₈ straight or branched alkoxy, and —OC(O)R₂ substituents are optionally substituted; and wherein one or more carbon atom(s) of said alkyl, alkenyl, alkynyl, or alkoxy, is optionally replaced with nitrogen, oxygen, or sulfur; or

R is phenyl optionally substituted with 1 to 5 substituents selected from the group consisting of hydroxy, carboxy, amino, halo, nitro, sulfhydryl, trifluoromethyl, methyl, C₁-C₈ straight or branched alkyl, C₂-C₈ straight or branched alkenyl or alkynyl, methoxy, and C₂-C₈ straight or branched alkoxy, wherein said hydroxy, carboxy, amino, sulfhydryl, halo, nitro, C₁-C₈ straight or branched alkyl, C₂-C₉ straight or branched alkenyl or alkynyl, methoxy, and C₂-C₈ straight or branched alkoxy substituents are optionally substituted; and wherein one or more carbon atom(s) of said alkyl, alkenyl, alkynyl, or alkoxy, is optionally replaced with nitrogen, oxygen, or sulfur; and

and wherein the stereochemistry at each of the 2, 4′, and 8′ positions is R or S.

### Formula II

The vitamin E derivative may be a compound of formula II:

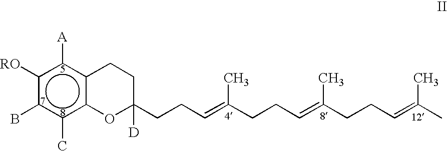

or a pharmaceutically acceptable salt, ester, or solvate thereof, wherein:

A, B, C, and D are independently hydrogen or methyl;

R is a reaction product derived from a reactant compound selected from the group consisting of (flava-3-ol)ₙ, wherein n is 1-12, α-ketoglutaric acid, α-butyric acid, β-Carotene, 1-methylinosine, 1-methylguanosine, 1-methyladenosine, 1-methylpseudouridine, 1,3-bisphosphoglycerate, 1,3-diphosphoglycerate, 2-methyladenosine, 2-methylthio-N6-isopentenyladenosine, 2-thiouridine, 2-thiocytidine, 2′-O-methylpseudouridine, 2′-O-methyluridine, 2′-O-methylcytidine, 2′-O-methyl-N2,N2-dimethyl-guanosine, 2′-O-methyl-5-methyluridine, 2′-O-methyl-1-methyladenosine, 2′-O-methylguanosine, 2′-O-methyl-N6-methyladenosine, 2′-O-methyladenosine, 2,6-di(tert-butyl)-4-methylphenol, 3-(3-amino-3-carboxypropyl)uridine, 3-carboxy-3-aminopropyl analogues, 3-methylcytidine, 3-methoxy-4-hydroxymandelic acid, 3-phosphoglycerate, 4-thiouridine, 5-carboxymethylaminomethyluridine, 5-methylaminomethyl-2-thiouridine, 5-carboxymethylaminomethyl-2-thiouridine, 5-methylcytidine, 5-methyluridine, 5-(carboxyhydroxymethyl)uridine methyl ester, 5-(carboxyhydroxymethyl)uridine, 5-carbamoylmethyluridine, 5-methyl-2-thiouridine, 5-methoxyuridine, 5-methoxycarbonylmethyl-2-thiouridine, 5-methylaminomethyluridine, 5-methyoxycarbonylmethyluridine, 5-phosphoribose-1-pyrophosphoric acid, 6-gingerol, 7-methylguanosine, acetyl-L-carnitine, acetylcholine, acyl gulycerols, ajoene, alanine, aminocyclopropane-carboxylic acid (ACC), anserine, anthocyanin, apigenin, arachidonic acid, arginine, astaxanthin, beta,D-galactosylqueuosine, beta,D-mannosylqueuosine, betaine, biopterin, biotin coenzymes such as carboxy biotin, biotin, calcium pectate, carbamyl phosphate, carnitine, carnosine, catechin, chlorogenic acid, cholesterol, choline, chondronectin, cis-zeatin, cis-aconitic acid, citric acid, Co-enzyme Q₁₀, cobamide coenzymes such as methylcobalamine or deoxyadenosylcobalamine, creatine, creatinine, cryptoxanthin, cumic acid, cumidine, curcumin, cyanidin chloride, cytochrome B, cytochrome B₅₅₈, cytochrome A, cytochrome C₁, cytochrome A₃, d-limonene, daidzein, diacylglycerol and its derivatives, dicetyl phosphate, diglycerides, dihydrouridine, dihydroxyacetone-phosphate, diphosphatidyl glycerol, diphthamide, dopamine, elastin, ellagic acid, epicatechin gallate, epicatechin, epigallocatechin, epigallocatechin gallate, epinephrine, erythrose-4-phosphate, ether phospholipids, farnesyl, fibronectins, fisetin, flavin coenzymes such as flavin mononucleotide or flavin adenine dinucleotide, flavoxanthine, folic acid, folic or pteridine coenzymes such as tetrahydrofolic acid, formic acid, fructose 1,6-bisphosphate, fructose-6-phosphate, fumaric acid, gallic acid, genistein, geranyl, ginkgolide A, ginkgolide B, ginkgolide C, glucose, glutamic acid, glutamine, glutathione, glyceraldehyde-3-phosphate, glycolipids, glycoproteins, GTP, hesperidin, histamine, histones, HMG Co-A, homoserine lactone, homoserine, indole-3-carbinol, inosinate, inosine, inositol, isocitric acid, isoleucine, kynurenine, L-histidine, L-dopa, laminin, lecithin, leucine, leukotrienes, linatine, lipoic coenzymes such as reduced or oxidized lipoamide, lipoic acid, lupeol, lutein, luteolin, lycopene, lycophyll, lycoxanthine, lysine, lysolecithin, lysophospholipids, malic acid, mandelic acid, melanins, melatonin, metanephrine, methionine, methylated estrogen, methylated lipids, methylated histones, methylated glycolipids, methylated sugars, methylated nucleic acids, methylated proteins, methylated ribosomal proteins, methylated lipids, methylated neurotransmitters, methylated glycoproteins, N-((9-beta-D-ribofuranosylpurine-6-yl)carbamoyl)-threoni ne, N-methylglycine, N-methyl histamine, N-malonyl ACC, N-((9-beta-D-ribofuranosylpurine-6-yl)n-methyl-carbamoyl) threonine, N-((9-beta-D-ribofuranosyl-2-methylthiopurine-2-yl)-carb amoyl)threonine, N-acetyl-neurominic acid, N2-(5-amino-5-carboxypentyl)cytidine, N2-methylguanosine, N2,N2-dimethlyguanosine, N4-acetylcytidine, N6-methyladenosine, N6-isopentenyladenosine, neopterin, nervonic acid, nicotinamide coenzymes such as nicotinamide adenine dinucleotide or nicotinamide adenine dinucleotide phosphate, nicotinic acid, N,N-dimethylglycine, N,N-dimethyltryptamine, norepinephrine, normetanephrine, nucleotide coenzymes such as UDP-glucose, CDP-choline, CDP-diacylglycerol, CMP-sialic acid, and other nucleotide derivatives of carbohydrates, alcohols, amino acids, lipids, or inorganic compounds, Omega-3 fatty acids, ornithine, oxaloacetic acid, p-coumaric acid, palmitic acid, pantothenic coenzymes such as pantothenic CoA, pantothenic dephospho-CoA, or 4-phosphopantothenate, pantothenic acid, para-aminobenzoic acid (PABA), pectin, phenylalanine, phosphatidyl ethanolamine, phosphatidyl choline, phosphocreatine, phosphoenol pyruvate, phosphonolipids, phytic acid, phytochlorin, phytol, picolinic acid, plasmalogens, plastoquinone, proanthocyanin, pseudouridine, pyridoxine coenzymes such as pyridoxal-phosphate or pyridoxamine-phosphate, pyruvate, pyruvate, quercetin, queuine, queuosine, quinolinic acid, ribose-5-phosphate, ribosomal proteins, ribulose-1,5-biphosphate, rutin, S-allymercaptocysteine, sarcosine, sedoheptulose-1,7-bisphosphate, sedoheptulose-7-phosphate, serotonin, sesamin, silybin, soy isoflavonoids, sphingolipids, sphingomyelin, sphingosine, steraylamine, succinic acid, sugars such as lactose, glucose, or mannose, sulforaphone, sulphorane, taurine, taxicatin, taxicin I, taxicin II, taxifolin, taxine A, taxodione, testosterone, tetrahydrobiopterin and its derivatives, tetrahydrofolate, thiamine coenzymes such as thiamine monophosphate, thiamine diphosphate, or thiamine triphosphate, threonine, trimethylysine, tryptamine, tryptophane, tumeric, tyrosine, uridine-5-oxyacetic acid, uridine-5-oxoacetic acid methyl ester, vaccenic acid, valine, vanillic acid, vitamin A, vitamin B₁, (Thiamine), vitamin B₂ (Riboflavin), vitamin B₆ (Pyrodoxine), vitamin B₁₂ (Cyanocobalamine), vitamin C (esterified or non-esterified Ascorbic Acid), vitamin D (Ergocalciferol), wybutosine, wybutoxosine, wye base, xanthophyll, xanthoxylin, xylulose-5-phosphate, and zeaxanthin;

R is a straight or branched C₁-C₃₀ alkyl, or C₂-C₃₀ straight or branched alkenyl or alkynyl optionally substituted with 1 to 12 substituents selected from the group consisting of hydroxy, carboxy, amino, halo, nitro, sulfhydryl, and J, wherein J is phenyl or a 5-7 membered heterocyclic ring which has one or more O, N, or S as the heteroatom(s), and J is optionally substituted with 1 to 5 substituents selected from the group consisting of hydroxy, carboxy, amino, halo, nitro, sulfhydryl, methyl, straight or branched C₂-C₁₀ alkyl, alkenyl, or alkynyl, methoxy, C₂-C₈ straight or branched alkoxy, and —OC(O)R₂ where R₂ is trifluoromethyl, methyl, straight or branched C₁-C₁₀ alkyl, or straight or branched C₂-C₁₀ alkenyl or alkynyl, wherein said hydroxy, carboxy, amino, halo, nitro, sulfhydryl, C₁-C₁₀ alkyl, C₂-C₁₀ alkenyl or alkynyl, methoxy, C₂-C₈ straight or branched alkoxy, and —OC(O)R₂ substituents are optionally substituted; and wherein one or more carbon atom(s) of said alkyl, alkenyl, alkynyl, or alkoxy, is optionally replaced with nitrogen, oxygen, or sulfur; or

R is phenyl optionally substituted with 1 to 5 substituents selected from the group consisting of hydroxy, carboxy, amino, halo, nitro, sulfhydryl, trifluoromethyl, methyl, C₁-C₈ straight or branched alkyl, C₂-C₈ straight or branched alkenyl or alkynyl, methoxy, and C₂-C₈ straight or branched alkoxy, wherein said hydroxy, carboxy, amino, sulfhydryl, halo, nitro, C₁-C₈ straight or branched alkyl, C₂-C₈ straight or branched alkenyl or alkynyl, methoxy, and C₂-C₈ straight or branched alkoxy substituents are optionally substituted; and wherein one or more carbon atom(s) of said alkyl, alkenyl, alkynyl, or alkoxy, is optionally replaced with nitrogen, oxygen, or sulfur;

and wherein the stereochemistry at each of the 2, 4, and 8′ positions is R or S.

### Methods of the Present Invention

The present invention relates to a method for preventing or treating diseases or conditions; effecting aging and longevity, nerve activity, hematopoiesis, blood chemistry, and blood cells, hepatic activity, nephritic activity, heart and cardiovascular function, pulmonary function, muscular function, cartilage, bone, and joint health, gastrointestinal function, reproductive system function, vision, immunoregulatory function, cell membrane integrity, and pain and inflammation; treating cancers or obesity; and in reducing the risk of Sudden Infant Death Syndrome in an animal, which comprises administering to said animal an effective amount of a vitamin E derivative compound of formula I or formula II.

In addition, the compounds and compositions of the present invention may be used to inhibit platelet aggregation, to decrease the release of superoxides by human peripheral blood neutrophils, to reduce the levels of tumor necrosis factor and interleukin-1, to increase antibody titers in the blood, or to treat, prevent, or delay the onset of one or more of the following diseases or conditions: immunoregulatory disease, inflammation, fever, edema, diabetes mellitus, cancer, signs of aging, pain, rheumatoid diseases, septic shock, chronic fatigue syndrome, and function laesa. Alternatively, they may be used to decrease Lipoprotein A concentrations in the blood.

Further, the compounds of the present invention are effective in decreasing Lipoprotein A concentration in the blood or, alternatively, in the treatment or prevention of one or more of the following diseases or conditions: immunoregulatory disease, inflammation, fever, edema, diabetes mellitus, cancer, signs of aging, pain, rheumatoid diseases, septic shock, chronic fatigue syndrome, and function laesa.

This invention includes several novel uses for tocopherol and tocotrienol conjugates. Examples include their use as antiinflammatory, antiatherogenic, and immunoregulatory agents, for the treatment of fever, edema, diabetes mellitus, cancer, signs of aging, pain, septic shock, chronic fatigue syndrome, and function laesa. In addition, tocopherol and tocotrienol conjugate compounds are useful for decreasing Lipoprotein A concentration in the blood, reducing total serum and LDL-cholesterol, apolipoprotein B, thromboxane A₂, platelet factor 4, triglycerides, and glucose, and inhibiting the activity of HMG-CoA reductase. They also reduce the levels of TNF and IL-1, and possibly IL-2 and γ-interferon, while increasing antibody titers in response to foreign proteins. In combination, these factors may produce a variety of advantageous and novel biological results, including a decrease in the release of superoxide and other cytotoxins produced by immunoregulatory cells and an increase in antibody titers.

In a preferred embodiment, the present invention further relates to a method for preventing or treating diseases or conditions selected from the group consisting of tissue damage resulting from physical trauma, tissue damage resulting from cell damage or cell death due to necrosis or apoptosis, neuronal mediated tissue damage or diseases, neural tissue damage resulting from ischemia and reperfusion injury, neurological disorders and neurodegenerative diseases, vascular stroke, cardiovascular disorders, age-related macular degeneration, AIDS and other immune diseases, arthritis, atherosclerosis, cachexia, cancer, degenerative diseases of skeletal muscle involving replicative senescence, diabetes, head trauma, immune senescence, inflammatory bowel disorders, muscular dystrophy, osteoarthritis, osteoporosis, chronic pain, acute pain, neuropathic pain, nervous insult, peripheral nerve injury, renal failure, retinal ischemia, septic shock, skin aging, altered circadian rhythmicicty, obesity, sickle cell anemia, cystic fibrosis, diseases or disorders relating to lifespan or proliferative capacity of cells, and diseases or disease conditions induced or exacerbated by cellular senescence, which comprises administering to said animal an effective amount of a vitamin E derivative compound of formula I or formula II.

Aging/longevity: oxidative stress, age-related memory impairment, hair loss,

In a preferred embodiment, the effect on nerve activity is selected from the group consisting of treating anxiety; treating depression; treating depression secondary to chronic diseases such as arthritis, fibromyalgia, liver disease, and alcoholism; treating dementia; treating schizophrenia; treating Alzheimer's disease; treating Parkinson's disease; treating demyelinating disorders; treating peripheral neuropathies; enhancing mood and behavior; maintaining or effecting neuronal membrane ratios of phosphatidyl choline and cholesterol; and effect on brain function, wherein the effect on brain function is selected from the group consisting of hyperbaric oxygen, neurotoxins, Alzheimer's disease, senile dementia, Parkinson's disease and MPTP, hypertensive cerebrovascular injury, cerebral trauma, neuronal ceroid lipofuscinoses, demyelinating diseases, ataxia-telangiectasia syndrome, potentiation of traumatic injury, and aluminum toxicity.

The compounds of this invention may also be used to alter the serum or plasma levels of several other blood constituents. For example, these compounds lower the plasma levels of thromboxane A₂ and platelet factor 4. In addition, they may serve passively as simple antioxidants or actively by decreasing the release of superoxides by neutrophils and other cytotoxins or cytokines, mast cells, macrophages, endothelial tissue, and other immunoregulatory tissues.

The thromboxanes (whose plasma levels are decreased using the compounds of this invention) also induce platelet aggregation and vasoconstriction. Therefore, the tocopherol and tocotrienol conjugate compounds of this invention may be used to reduce blood clotting in a wide variety of applications. For example, these compounds may be used to treat or prevent diseases, such as thrombotic diseases, cardiovascular diseases, hypertension, pulmonary diseases, and renal diseases. Specifically, the compounds of this invention may be used to prevent or reverse blood clots and lesions which may cause diseases such as myocardial infarction, stroke, pulmonary embolism, deep vein thrombosis, peripheral arterial occlusion, and other blood system thromboses.

HMG-CoA reductase catalyzes the rate-limiting step of cholesterol biosynthesis. Therefore, a reduction in its activity decreases the total amount of cholesterol in the serum of animals and humans alone, or in combination with a low fat, low cholesterol diet. The effects are most noticeable in hypercholesterolemic individuals with poor dietary regimens.

Blood disorders: lead poisoning, protoporphyrin photooxidation, malaria, sickle-cell anemia, favism, fanconi anemia, hematopoiesis,

In another preferred embodiment, the effect on hepatic activity is selected from the group consisting of treating cirrhosis, chronic liver disease, alcoholic liver damage, toxic chemical exposure, non-steroidal anti-inflammatory drug-related liver damage, estrogen induced liver problems, bile disorders, and environmental chemical hypersensitivity. Liver disorders: endotoxin liver injury, carbon tetrachloride liver injury,

Kidney disorders: nephritic antiaglomerular basement membrane disease, aminoglycoside nephrotoxicity, heavy metal nephrotoxicity, renal graft rejection,

In another preferred embodiment, the effect on heart and cardiovascualr function is treating or reducing heart and/or artery disease risk due to elevated blood levels of homocysteine, diseases associated with high levels of cholesterol, alcohol cardiomyopathy, Keshan disease, atherosclerosis, doxorubicon toxicity, peripheral circulation problems, stroke, management of LDL oxidation, coronary heart disease, myocardial infarction, and ischemic heart disease.

Because of their ability to lower total serum cholesterol and low density lipoprotein-cholesterol and increase the HDL-/LDL-cholesterol ratio, the novel tocopherol and tocotrienol conjugate compounds of this invention may be used in the prevention and treatment of diseases associated with high levels of cholesterol. In another preferred embodiment, diseases associated with high levels of cholesterol that may be treated by the compounds of this invention include, but are not limited to, atherosclerosis, thrombosis, coronary artery disease, and other forms of cardiovascular disease. In addition, the ability of the compounds of this invention to lower serum glucose levels may increase the insulin production in Type 2 diabetics.

Hypercholesterolemic diseases and conditions that may be treated using the compositions and mixtures described herein include, but are not limited to, arteriosclerosis, atherosclerosis, xanthomatosis, hyperlipoproteinemias, and familial hypercholesterolemia.

Antiatherogenic diseases and conditions that may be treated using such compositions include, but are not limited to, arteriosclerosis, atherosclerosis, myocardial infarction, ischemia (i.e., myocardial, brain, and renal ischemia), and strokes.

The tocopherol and tocotrienol conjugate compounds of the present invention are also capable of acting as antiatherogenic agents by inhibiting or reversing the oxidation of LDL and by protecting vascular tissue in general from oxidative damages. The oxidated form of LDL (“OX-LDL”) is a major component in the formation of atheroma. Such formation commonly results in a narrowing of the arteries by atherosis plaques. While not wishing to be bound by theory, we believe that by reducing serum LDL levels, tocopherol and tocotrienol conjugate compounds enhance the rate of metabolic LDL turnover and therefore, decrease the exposure of LDL to oxidative agents.

Tocopherol and tocotrienol conjugate compounds also decrease the concentration of lipoprotein A in the blood. It has been well established that elevated concentrations of Lipoprotein A are correlated with early onset and progression of atherosclerosis, premature myocardial ischemia, and rheumatoid arthritis. In fact, Lipoprotein A concentration is a more accurate indicator of coronary heart disease than LDL concentration. Lowering Lipoprotein A concentrations in individuals having high Lipoprotein A levels (above about 20 mg/dl) drastically reduces the probability of atherosclerosis and coronary heart disease.

Tocopherol and tocotrienol conjugate compounds also exhibit diuretic activity—they are antagonistic to vasopressin and angiotensin II. Accordingly, these compounds are useful in the treatment and management, for example, of hypertension.

cardiac ischemia, microembolic and/or frank occlusion, reocclusion following transluminal angioplasty, myocardial infarction, cardiopulmonary bypass associated dysfunction, peripheral vasoconstriction, organ dysfunction, platelet consumption and/or activation (and subsequent decreased function, aggregation, and decreased numbers), mitral valve pathology associated with acute perioperative pulmonary hypertension, chronic obstructive arterial disease caused by arteriosclerosis, Raynaud's syndrome, vasoconstriction, renal artery stenosis, myocardial infarction, stroke, deep vein thrombosis, peripheral arterial occlusion, and other blood system thromboses.

Pulmonary diseases and conditions that may be treated using such compositions include, but are not limited to, pulmonary disease, in general (such as reduced specific conductance, reduced dynamic compliance and constriction (contraction of smooth muscle), excess pulmonary fluids (such as pulmonary lymph, foam, or bronchoalveolar lavage), adult respiratory distress syndrome, astatis and rhinitic disease (such as pulmonary and systemic hypertension, pulmonary edema, fluid accumulation/neutrophil infiltration, and pulmonary vascular permeability), pulmonary vasoconstriction (associated, for example, with endotoxemia, gram-negative organisms, anaphylaxis, hemorrhagic shock, or allergy to ragweed), and pulmonary embolism. Lung: effects of cigarette smoke, emphysema, hyperoxia, bronchopulmonary dysplasia, oxidant pollutants, acute respiratory distress syndrome, pneumonoconiosis, bleomycin toxicity, paraquat toxicity,

In a further preferred embodiment, the effect on cartilage, bone and joint health is selected from the group consisting of treating osteoarthritis, rheumatoid arthritis, fibromyalgia, joint injuries, joint inflammation, joint degeneration, and osteoporosis.

Gastrointestinal disorders: alloxan action, free fatty acid-induced pancreatitis, nonsteroidal anti-inflammatory drug-induced lesions, abetalipoproteinemia, Inhibition of formation of carcinogenic nitrosamines

Reproductive disorders: spontaneous abortion, infertility/sterility, sexual performance, post-menopausal syndrome, prostaglandin disorders,

Muscular system: necrotizing myopathy, muscular dystrophy,

Eye/vision: cataracts, age-related macular degeneration, ocular hemorrhage, degenerative retinal damage, retinopathy,

Immunoregulatory diseases and diseases that may be treated using the compositions of this invention include, but are not limited to, chronic fatigue syndrome, graft rejections, autoimmune diseases, such as AIDS, and other viral diseases that weaken the immune system.

The tocopherol and tocotrienol conjugate compounds of this invention and mixtures thereof have also been found to reduce the levels of tumor necrosis factor in response to lipopolysaccharide stimulation, lower arachidonic acid in the tissues, and reduce oxygen metabolites in the blood of animals and humans. These results point to an overall reduction in prostaglandins and leukotrienes, both of which are synthesized from arachidonic acid, and a possible reduction in interleukin-1. Accordingly, the compounds of this invention may be employed for a variety of uses. For example, they may be used to prevent endothelial injury, such as ischemic and reperfused myocardium and ulcers. In addition, the inhibition of tumor necrosis factor biosynthesis would also be accompanied by a decrease in inflammation—i.e., through inhibiting the respiratory bursts of neutrophils or through free radical scavenging. Therefore, the compounds of this invention are also useful as antiinflammatory agents for the prevention and treatment of a wide variety of diseases and conditions involving minor, acute, and chronic inflammation. These include, but are not limited to, fever, rheumatoid diseases, pain, function laesa, hypertension, and edema.

Inflammatory diseases and conditions that may be treated using such compositions include, but are not limited to, essential hypertension, hypertension of congestive heart failure, renal dysfunction caused by reduced myocardia output, endotoxemia, chronic liver disease or hypertension, pulmonary inflammation in asthma, bronchitic, pneumonia or acute lung injury, rheumatic diseases (such as rheumatoid arthritis and systemic lupus crythematosus), inflammatory bowel disease (such as ulcerative colitis), irritable bowel disease (such as villous adenoma), gastrointestinal disorders caused by excess acids, pepsin, or bile salts, Zollinger-Ellison syndrome, skin diseases or trauma (such as burns, acid, or caustic injury), gout, Bartter's syndrome, fever, rheumatoid diseases, pain, function laesa, hypertension, and edema.

Preferred antioxidizing uses include, but are not limited to, the treatment and prevention of endothelial injury, such as ischemic and reperfused myocardium. Because of their antioxidizing activity, the tocopherol and tocotrienol conjugate compounds of this invention may also be used in treating and preventing cancer, such as prostate cancer, for example by preventing cancer-causing mutations in the DNA of an animal.

The tocopherol and tocotrienol conjugate compounds of this invention may be altered by known chemical means to produce various derivatives or analogues. Such derivatives or analogues may be more easily isolated in pure form, or more resistant to degradation, or possess other desired characteristics. Such derivatives and analogues are also envisioned by this invention. This invention encompasses the d- or l-isomer and the d,l-racemic mixture of each tocopherol and tocotrienol compound. However, the naturally occurring d-isomer is preferred. This invention also includes mixtures of at least one novel tocopherol or tocotrienol conjugate compound of this invention with one or more other compound(s).

### Pharmaceutical Compositions of the Present Invention

The present invention also relates to a pharmaceutical composition comprising:

(i) an effective amount of a vitamin E derivative compound of formula I or formula II; and

(ii) a pharmaceutically acceptable carrier.

The compounds and mixtures described herein are useful in pharmaceutical compositions and dietary supplements. Advantageously, these products are hypocholesterolemic, antithrombotic, antioxidizing, antiatherogenic, anti-inflammatory, or immunoregulatory agents.

Tocopherol and tocotrienol conjugate compounds and mixtures thereof may also be used in combination with conventional therapeutics used in the treatment or prophylaxis of any of the aforementioned diseases. Such combination therapies advantageously utilize lower dosages of those conventional therapeutics, thus avoiding possible toxicity incurred when those agents are used as monotherapies. For example, tocopherol and tocotrienol compounds may be used in combination with bile acid sequestrants, such as Cholestyramine and Colestipol; fibric acid derivatives, such as, Clofibrate, Gamfibrozil, Bezafibrate, Fenofibrate, and Ciprofibrate; HMGR inhibitors, such as Lovastatin, Mevastatin, Pravastatin, Simvastatin, and SRI-62320; Probucol; Nicotinic Acid; its derivatives and conjugates, such as, 6-OH-Nicotinic Acid, Nicotinaria Acid, Nicotinamide, Nicotinamide-N-oxide, 6-OH-Nictinamide, NAD, N-Methyl-2-pyridine-8-carboxamide, N-Methyl-Nicotinamide, N-Ribosyl-2-Pyridone-S-Carboxide, N-Methyl-4-pyridone-5-carboxamide, Bradilian, Niceritrol, Sorbinicate, and Hexanicit; Neomycin and d-Thyroxine.

The novel pharmaceutical compositions of the invention include a therapeutically effective amount of the active agent indicated above. This effective amount will generally comprise from about 0.1 mg to about 100 mg of the active agent per kilogram of patient body weight per day. This effective amount can vary depending upon the physical status of the patient and other factors well known in the art. Moreover, it will be understood that this dosage of active agent can be administered in a single or multiple dosage units to provide the desired therapeutic effect. If desired, other therapeutic agents can be employed in conjunction with those provided by the present invention.

Pharmaceutical compositions may take the form of tablets, capsules, emulsions, suspensions, and powders for oral administration, sterile solutions or emulsions for parenteral administration, sterile solutions for intravenous administration and gels, lotions and cremes for topical application. The pharmaceutical compositions may be administered to an animal in need thereof in a safe and pharmaceutically effective amount to elicit any of the desired results indicated for the compounds and mixtures described herein.

The compounds of the invention are preferably delivered to the patient by means of a pharmaceutically acceptable carrier. Such carriers are well known in the art and generally will be in either solid or liquid form. Solid form pharmaceutical preparations which may be prepared according to the present invention include powders, tablets, dispersible granules, capsules, cachets and suppositories. In general, solid form preparations will comprise from about 5% to about 90% by weight of the active agent.

A solid carrier can be one or more substances which may also act as diluents, flavoring agents, solubilizers, lubricants, suspending agents, binders or tablet disintegrating agents; it can also be encapsulating material. In powders, the carrier is a finely divided solid which is in admixture with the viscous active compound. In tablets, the active compound is mixed with a carrier having the necessary binding properties in suitable proportions and compacted to the shape and size desired. Suitable solid carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating materials as a carrier which may provide a capsule in which the active component (with or without other carriers) is surrounded by carrier, which is thus in association with it. Similarly, cachets are included. Tablets, powders, cachets, and capsules can be used as solid dosage forms suitable for oral administration. If desired for reasons of convenience or patient acceptance, pharmaceutical tablets prepared according to the invention may be provided in chewable form, using techniques well known in the art.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby to solidify.

Liquid form preparations include solutions, suspensions, and emulsions. As an example may be mentioned water or water/propylene glycol solutions for parenteral injection. Liquid preparations can also be formulated in solution in aqueous polyethylene glycol solution. Aqueous solutions suitable for oral use can be prepared by dissolving the active component in water and adding suitable colorants, flavors, stabilizers, and thickening agents as desired. Aqueous suspensions suitable for oral use can be made my dispersing the finely divided active component in water with a viscous material, i.e., natural or synthetic gums, resins, methylcellulose, sodium carboxymethylcellulose, and other well known suspending agents. Liquid pharmaceutical preparations may comprise up to 100% by weight of the subject active agent.

Also contemplated as suitable carriers are solid form preparations which are intended to be converted, shortly before use, to liquid form preparations for either oral or parenteral administration. Such liquid forms include solutions, suspensions, and emulsions. These particular solid form preparations are most conveniently provided in unit dose form and as such are used to provide a single liquid dosage unit. Alternately, sufficient solid may be provided so that after conversion to liquid form, multiple individual liquid doses may be obtained by measuring predetermined volumes of the liquid form preparation as with a syringe, teaspoon, or other volumetric container. When multiple liquid doses are so prepared, it is preferred to maintain the unused portion of said liquid doses at low temperature (i.e., under refrigeration) in order to retard possible decomposition. The solid form preparations intended to be converted to liquid form may contain, in addition to the active material, flavorants, colorants, stabilizers, buffers, artificial and natural sweeteners, dispersants, thickeners, solubilizing agents, and the like. The liquid utilized for preparing useful liquid form preparations may be water, isotonic water, ethanol, glycerine, propylene glycol, and the like as well as mixtures thereof. Naturally, the liquid utilized will be chosen with regard to the route of administration. For example, liquid preparations containing large amounts of ethanol are not suitable for parenteral use.

The pharmaceutical preparation may also be in a unit dosage form. In such form, the preparation is subdivided into unit doses containing appropriate quantities of the active component. The unit dosage form can be a packaged preparation, the package containing discrete quantities of preparation, for example, packeted tablets, capsules, and powders in vials or ampoules. The unit dosage form can also be a capsule, cachet, or tablet itself or it can be the appropriate number of any of these in packaged form.

The pharmaceutical preparations of the invention may include one or more preservatives well known in the art, such as benzoic acid, sorbic acid, methylparaben, propylparaben and ethylenediaminetetraacetic acid (EDTA). Preservatives are generally present in amounts up to about 1% and preferably from about 0.05 to about 0.5% by weight of the pharmaceutical composition.

Useful buffers for purposes of the invention include citric acid-sodium citrate, phosphoric acid-sodium phosphate, and acetic acid-sodium acetate in amounts up to about 1% and preferably from about 0.05 to about 0.5% by weight of the pharmaceutical composition. Useful suspending agents or thickeners include cellulosics like methylcellulose, carageenans like alginic acid and its derivatives, xanthan gums, gelatin, acacia, and microcrystalline cellulose in amounts up to about 20% and preferably from about 1% to about 15% by weight of the pharmaceutical composition.

Sweeteners which may be employed include those sweeteners, both natural and artificial, well known in the art. Sweetening agents such as monosaccharides, disaccharides and polysaccharides such as xylose, ribose, glucose, mannose, galactose, fructose, dextrose, sucrose, maltose, partially hydrolyzed starch or corn syrup solids and sugar alcohols such as sorbitol, xylitol, mannitol, and mixtures thereof may be utilized in amounts from about 10% to about 60% and preferably from about 20% to about 50% by weight of the pharmaceutical composition. Water soluble artificial sweeteners such as saccharin and saccharin salts such as sodium or calcium, cyclamate salts, acesulfame-K, aspartame, and the like, and mixtures thereof

may be utilized in amounts from about 0.001% to about 5% by weight of the composition.

Flavorants which may be employed in the pharmaceutical products of the invention include both natural and artificial flavors, and mints such as peppermint, menthol, vanilla, artificial vanilla, chocolate, artificial chocolate, cinnamon, various fruit flavors, both individually and mixed, in amounts from about 0.5% to about 5% by weight of the pharmaceutical composition.

Colorants useful in the present invention include pigments which may be incorporated in amounts of up to about 6% by weight of the composition. A preferred pigment, titanium dioxide, may be incorporated in amounts up to about 1%. Also, the colorants may include other dyes suitable for food, drug, and cosmetic applications, known as F.D.&C. dyes and the like. Such dyes are generally present in amounts up to about 0.25% and preferably from about 0.05% to about 0.2% by weight of the pharmaceutical composition. A full recitation of all F.D.&C. and D.&C. dyes and their corresponding chemical structures may be found in the Kirk-Othmer Encyclopedia of Chemical Technology, in Volume 5, at pages 857-884, which text is accordingly incorporated herein by reference.

Useful solubilizers include alcohol, propylene glycol, polyethylene glycol, and the like, and may be used to solubilize the flavors. Solubilizing agents are generally present in amounts up to about 10%; preferably from about 2% to about 5% by weight of the pharmaceutical composition.

Lubricating agents which may be used when desired in the instant compositions include silicone oils or fluids such as substituted and unsubstituted polysiloxanes, e.g., dimethyl polysiloxane, also known as dimethicone. Other well known lubricating agents may be employed.

It is not expected that compounds of the present invention will display significant adverse interactions with other synthetic or naturally occurring substances. Thus, a vitamin E derivative compound of the present invention may be administered in combination with other compounds and compositions useful for preventing or treating diseases or conditions; effecting aging and longevity, nerve activity, hematopoiesis, and maintenance of blood cells, hepatic activity, nephritic activity, heart and cardiovascular function, pulmonary function, muscular function, cartilage, bone, and joint health, gastrointestinal function, reproductive system function, vision, immune function, cell membrane integrity, and pain and inflammation; preventing or treating diseases or conditions; treating cancers or obesity; and in reducing the risk of Sudden Infant Death Syndrome in an animal. In particular the compounds of the present invention may be administered in combination with other compounds of the present invention; other orthomolecular substances; vitamin(s) and/or cofactor(s) selected from the group consisting of vitamin A, β-Carotene, vitamin B₁ (Thiamine), vitamin B₂ (Riboflavin), vitamin B₆ (Pyrodoxine), biotin, inositol, folic acid, vitamin B₁₂ (Cyanocobalamine), nicotinic acid, vitamin C (esterified or non-esterified Ascorbic Acid), vitamin D (Ergocalciferol), pantothenic acid, phosphatidyl choline, tetrahydrofolate, tetrahydrobiopterin, β-tocopherol, γ-tocopherol, δ-tocopherol, α-tocotrienol, β-tocotrienol, γ-tocotrienol, δ-tocotrienol, para-aminobenzoic acid (PABA), and inosinate; other known over-the-counter and/or prescription drugs; and other compounds and compositions useful for preventing or treating diseases or conditions; effecting aging and longevity, nerve activity, hematopoiesis and maintenance of blood cells, hepatic activity, nephritic activity, heart and cardiovascular function, pulmonary function, muscular function, cartilage, bone, and joint health, gastrointestinal function, reproductive system function, vision, immune function, cell membrane integrity, and pain and inflammation; preventing or treating diseases or conditions; treating cancers or obesity; and in reducing the risk of Sudden Infant Death Syndrome in an animal.

The optimal pharmaceutical formulations will be determined by one skilled in the art depending upon considerations such as the route of administration and desired dosage. See, for example, “Remington's Pharmaceutical Sciences”, 18th ed. (1990, Mack Publishing Co., Easton, Pa. 18042), pp. 1435-1712, the disclosure of which is hereby incorporated by reference. Such formulations may influence the physical state, stability, rate of in vivo release, and rate of in vivo clearance of the present therapeutic agents of the invention.

### Synthesis of Vitamin E Derivatives

The compounds of the present invention may be readily prepared by standard techniques of organic chemistry, utilizing the general synthetic pathways depicted below.

In the preparation of the compounds of the invention, one skilled in the art will understand that one may need to protect or block various reactive functionalities on the starting compounds or intermediates while a desired reaction is carried out on other portions of the molecule. After the desired reactions are complete, or at any desired time, normally such protecting groups will be removed by, for example, hydrolytic or hydrogenolytic means. Such protection and deprotection steps are conventional in organic chemistry. One skilled in the art is referred to. “Protective Groups in Organic Chemistry,” McOmie, ed., Plenum Press, New York, N.Y.; and “Protective Groups in Organic Synthesis,” Greene, ed., John Wiley & Sons, New York, N.Y. (1981) for the teaching of protective groups which may be useful in the preparation of compounds of the present invention.

The product and intermediates may be isolated or purified using one or more standard purification techniques, including, for example, one or more of simple solvent evaporation, recrystallization, distillation, sublimation, filtration, chromatography, including thin-layer chromatography, HPLC (e.g. reverse phase HPLC), column chromatography, flash chromatography, radial chromatography, trituration, and the like.

### Scheme I

As depicted by Scheme I, a reaction product of α-butyric acid, α-tocotrienol, β-Carotene, β-tocopherol, β-tocotrienol, γ-tocopherol, γ-tocotrienol, δ-tocopherol, δ-tocotrienol, 3-carboxy-3-aminopropyl analogues, 3-methoxy-4-hydroxymandelic acid, 5-phosphoribose-1-pyrophosphoric acid, 6-gingerol, acetyl-L-carnitine, acetylcholine, ajoene, aminocyclopropane-carboxylic acid (ACC), anserine, anthocyanin, apigenin, arachidonic acid, astaxanthin, betaine, biopterin, Biotin, calcium pectate, carbamyl phosphate, carnitine, carnosine, catechin, chlorogenic acid, choline, Co-enzyme Q₁₀, creatine, creatinine, cryptoxanthin, cumic acid, cumidine, curcumin, cyanidin chloride, d-limonene, daidzein, diacylglycerol, diphosphatidyl glycerol, dopamine, ellagic acid, epicatechin, epicatechin gallate, epigallocatechin, epigallocatechin gallate, epinephrine, farnesyl, fibronectins, fisetin, (flava-3-ol)ₙ, wherein n is 1-12, flavoxanthine, Folic Acid, fructose 1,6-bisphosphate, gallic acid, genistein, geranyl, ginkgolide A, ginkgolide C, ginkgolide B, glucose, glutathione, GTP, hesperidin, hesperitin, histamine, HMG Co-A, homoserine lactone, indole-3-carbinol, inosinate, inositol, kynurenine, L-histidine, L-dopa, linatine, lipoic acid, lupeol, lutein, luteolin, lycophyll, lycopene, lycoxanthine, lysine, lysolecithin, mandelic acid, melanins, melatonin, metanephrine, methylated estrogen, methylated lipids, N-methylglycine, N-malonyl ACC, N-methyl histamine, neopterin, nervonic acid, nicotinic acid, N,N-dimethyltryptamine, N,N-dimethylglycine, norepinephrine, normetanephrine, omega-3 fatty acids, ornithine, p-coumaric acid, pantothenic acid, para-aminobenzoic acid (PABA), pectin, phosphatidyl choline, phosphatidyl ethanolamine, phosphocreatine, phytic acid, phytochlorin, phytol, picolinic acid, proanthocyanin, pyruvate, quercetin, queuine, queuosine, quinolinic acid, rutin, S-allymercaptocysteine, sarcosine, serotonin, sesamin, silybin, soy isoflavonoids, sulphorane, taurine, taxicatin, taxicin II, taxicin I, taxifolin, taxine A, taxodione, tetrahydrobiopterin and derivatives, tetrahydrobiopterin, tetrahydrofolate, trimethylysine, tryptamine, tumeric, vaccenic acid, vanillic acid, vitamin B₆ (Pyrodoxine), vitamin B₂ (Riboflavin), vitamin A, vitamin D (Ergocalciferol), vitamin C (esterified or non-esterified Ascorbic Acid), vitamin B₁₂ (Cyanocobalamine), vitamin B₁ (Thiamine), wye base and diphthamide, xanthophyll, xanthoxylin, or zeaxanthin bearing one or more free functional groups, such as hydroxyl and/or amine functional group(s), may be covalently attached by to a tocopherol molecule to produce a compound of formula I:

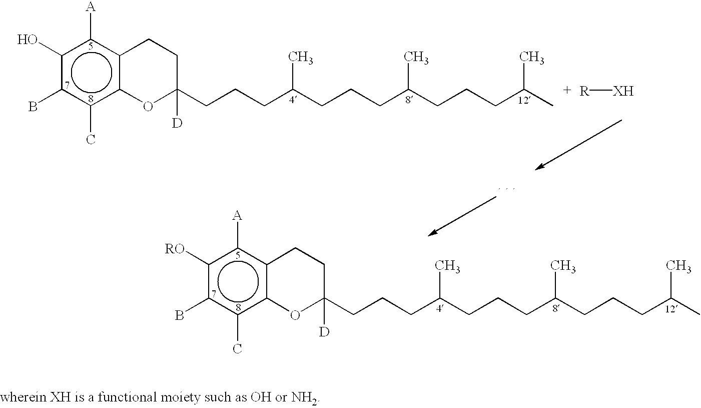

As depicted by Scheme II, a reaction product of α-butyric acid, α-tocotrienol, β-Carotene, β-tocopherol, δ-tocotrienol, γ-tocopherol, γ-tocotrienol, δ-tocopherol, δ-tocotrienol, 3-carboxy-3-aminopropyl analogues, 3-methoxy-4-hydroxymandelic acid, 5-phosphoribose-1-pyrophosphoric acid, 6-gingerol, acetyl-L-carnitine, acetylcholine, ajoene, aminocyclopropane-carboxylic acid (ACC), anserine, anthocyanin, apigenin, arachidonic acid, astaxanthin, betaine, biopterin, Biotin, calcium pectate, carbamyl phosphate, carnitine, carnosine, catechin, chlorogenic acid, choline, Co-enzyme Q₁₀, creatine, creatinine, cryptoxanthin, cumic acid, cumidine, curcumin, cyanidin chloride, d-limonene, daidzein, diacylglycerol, diphosphatidyl glycerol, dopamine, ellagic acid, epicatechin, epicatechin gallate, epigallocatechin, epigallocatechin gallate, epinephrine, farnesyl, fibronectins, fisetin, (flava-3-ol)ₙ, wherein n is 1-12, flavoxanthine, Folic Acid, fructose 1,6-bisphosphate, gallic acid, genistein, geranyl, ginkgolide A, ginkgolide C, ginkgolide B, glucose, glutathione, GTP, hesperidin, hesperitin, histamine, HMG Co-A, homoserine lactone, indole-3-carbinol, inosinate, inositol, kynurenine, L-histidine, L-dopa, linatine, lipoic acid, lupeol, lutein, luteolin, lycophyll, lycopene, lycoxanthine, lysine, lysolecithin, mandelic acid, melanins, melatonin, metanephrine, methylated estrogen, methylated lipids, N-methylglycine, N-malonyl ACC, N-methyl histamine, neopterin, nervonic acid, nicotinic acid, N,N-dimethyltryptamine, N,N-dimethylglycine, norepinephrine, normetanephrine, omega-3 fatty acids, ornithine, p-coumaric acid, pantothenic acid, para-aminobenzoic acid (PABA), pectin, phosphatidyl choline, phosphatidyl ethanolamine, phosphocreatine, phytic acid, phytochlorin, phytol, picolinic acid, proanthocyanin, pyruvate, quercetin, queuine, queuosine, quinolinic acid, rutin, S-allymercaptocysteine, sarcosine, serotonin, sesamin, silybin, soy isoflavonoids, sulphorane, taurine, taxicatin, taxicin II, taxicin I, taxifolin, taxine A, taxodione, tetrahydrobiopterin and derivatives, tetrahydrobiopterin, tetrahydrofolate, trimethylysine, tryptamine, tumeric, vaccenic acid, vanillic acid, vitamin B₆ (Pyrodoxine), vitamin B₂ (Riboflavin), vitamin A, vitamin D (Ergocalciferol), vitamin C (esterified or non-esterified Ascorbic Acid), vitamin B₁₂ (Cyanocobalamine), vitamin B₁ (Thiamine), wye base and diphthamide, xanthophyll, xanthoxylin, or zeaxanthin bearing one or more free functional groups, such as hydroxyl and/or amine functional group(s), may be covalently attached by to a tocopherol molecule to produce a compound of formula II:

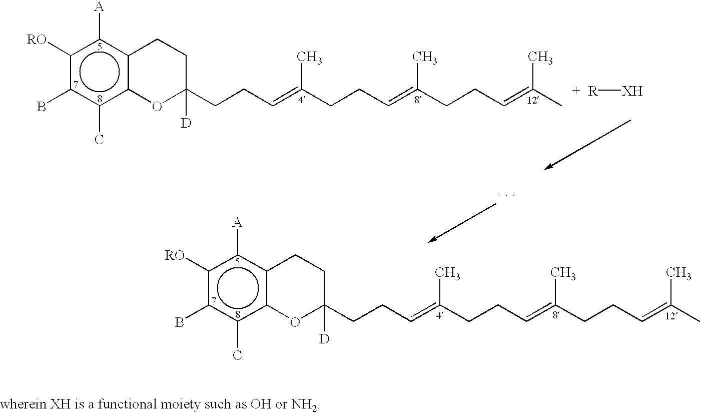

A representative example of the synthesis of a compound of formula I, covalently linking a reaction product of α-butyric acid, α-tocotrienol, β-Carotene, β-tocopherol, β-tocotrienol, γ-tocopherol, γ-tocotrienol, δ-tocopherol, δ-tocotrienol, 3-carboxy-3-aminopropyl analogues, 3-methoxy-4-hydroxymandelic acid, 5-phosphoribose-1-pyrophosphoric acid, 6-gingerol, acetyl-L-carnitine, acetylcholine, ajoene, aminocyclopropane-carboxylic acid (ACC), anserine, anthocyanin, apigenin, arachidonic acid, astaxanthin, betaine, biopterin, Biotin, calcium pectate, carbamyl phosphate, carnitine, carnosine, catechin, chlorogenic acid, choline, Co-enzyme Q₁₀, creatine, creatinine, cryptoxanthin, cumic acid, cumidine, curcumin, cyanidin chloride, d-limonene, daidzein, diacylglycerol, diphosphatidyl glycerol, dopamine, ellagic acid, epicatechin, epicatechin gallate, epigallocatechin, epigallocatechin gallate, epinephrine, farnesyl, fibronectins, fisetin, (flava-3-ol)ₙ, wherein n is 1-12, flavoxanthine, Folic Acid, fructose 1,6-bisphosphate, gallic acid, genistein, geranyl, ginkgolide A, ginkgolide C, ginkgolide B, glucose, glutathione, GTP, hesperidin, hesperitin, histamine, HMG Co-A, homoserine lactone, indole-3-carbinol, inosinate, inositol, kynurenine, L-histidine, L-dopa, linatine, lipoic acid, lupeol, lutein, luteolin, lycophyll, lycopene, lycoxanthine, lysine, lysolecithin, mandelic acid, melanins, melatonin, metanephrine, methylated estrogen, methylated lipids, N-methylglycine, N-malonyl ACC, N-methyl histamine, neopterin, nervonic acid, nicotinic acid, N,N-dimethyltryptamine, N,N-dimethylglycine, norepinephrine, normetanephrine, omega-3 fatty acids, ornithine, p-coumaric acid, pantothenic acid, para-aminobenzoic acid (PABA), pectin, phosphatidyl choline, phosphatidyl ethanolamine, phosphocreatine, phytic acid, phytochlorin, phytol, picolinic acid, proanthocyanin, pyruvate, quercetin, queuine, queuosine, quinolinic acid, rutin, S-allymercaptocysteine, sarcosine, serotonin, sesamin, silybin, soy isoflavonoids, sulphorane, taurine, taxicatin, taxicin II, taxicin I, taxifolin, taxine A, taxodione, tetrahydrobiopterin and derivatives, tetrahydrobiopterin, tetrahydrofolate, trimethylysine, tryptamine, tumeric, vaccenic acid, vanillic acid, vitamin B₆ (Pyrodoxine), vitamin B₂ (Riboflavin), vitamin A, vitamin D (Ergocalciferol), vitamin C (esterified or non-esterified Ascorbic Acid), vitamin B₁₂ (Cyanocobalamine), vitamin B₁ (Thiamine), wye base and diphthamide, xanthophyll, xanthoxylin, or zeaxanthin B₁ (Thiamine) with d-α-tocotrienol, is depicted in Scheme III:

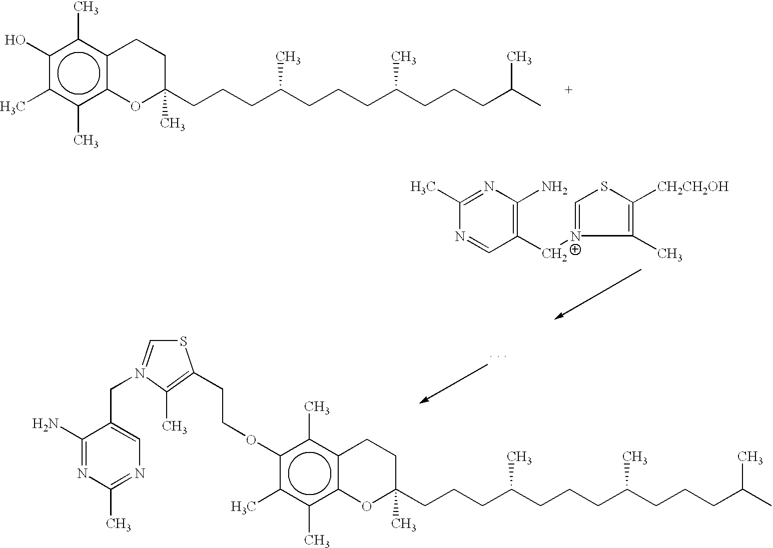

The route(s) of administration of the compounds and compositions of the present invention are well known to those skilled in the art (see, for example, “Remington's Pharmaceutical Sciences”, 18th Edition, Chapter 86, pp. 1581-1592, Mack Publishing Company, 1990). The compounds and compositions may be administered orally, parenterally, by inhalation spray, topically, rectally, nasally, buccally, vaginally, or via an implanted reservoir in dosage formulations containing conventional non-toxic pharmaceutically-acceptable carriers, adjuvants, and vehicles. The term parenteral as used herein includes subcutaneous, intravenous, intramuscular, intraperitoneally, intrathecally, intraventricularly, intrasternal, and intracranial injection or infusion techniques.

To be effective therapeutically as central nervous system targets, the compounds and compositions should readily penetrate the blood-brain barrier when peripherally administered. Compounds which cannot penetrate the blood-brain barrier can be effectively administered by an intraventricular route.

The compounds and compositions may be administered in the form of sterile injectable preparations, for example, as sterile injectable aqueous or oleaginous suspensions. These suspensions, may be formulated according to techniques known in the art using suitable dispersing or wetting agents and suspending agents. The sterile injectable preparations may also be sterile injectable solutions or suspensions in non-toxic parenterally-acceptable diluents or solvents, for example, as solutions in 1,3-butanediol. Among the acceptable vehicles and solvents that may be employed are water, Ringer's solution and isotonic sodium chloride solution. In addition, sterile, fixed oils are conventionally employed as solvents or suspending mediums. For this purpose, any bland fixed oil such as a synthetic mono- or di-glyceride may be employed. Fatty acids such as oleic acid and its glyceride derivatives, including olive oil and castor oil, especially in their polyoxyethylated versions, are useful in the preparation of injectables. These oil solutions or suspensions may also contain long-chain alcohol diluents or dispersants.

Additionally, in a preferred embodiment, the compounds and compositions may be administered orally in the form of capsules, tablets, aqueous suspensions, or solutions. Tablets may contain carriers such as lactose and corn starch, and/or lubricating agents such as magnesium stearate. Capsules may contain diluents including lactose and dried corn starch. Aqueous suspensions may contain emulsifying and suspending agents combined with the active ingredient. The oral dosage forms may further contain sweetening, flavoring, coloring agents, or combinations thereof. Delivery in an enterically coated tablet, caplet, or capsule, to further enhance stability and provide release in the intestinal tract to improve absorption, is the best mode of administration currently contemplated.

The compounds may also be administered rectally in the form of suppositories. These compositions can be prepared by mixing the drug with a suitable non-irritating excipient which is solid at room temperature, but liquid at rectal temperature and, therefore, will melt in the rectum to release the drug. Such materials include cocoa butter, beeswax, and polyethylene glycols.

Furthermore, the compounds may be administered topically, especially when the conditions addressed for treatment involve areas or organs readily accessible by topical application, including the lower intestinal tract. Suitable topical formulations can be readily prepared for such areas or organs. For example, topical application to the lower intestinal tract can be effected in a rectal suppository formulations (see above) or in suitable enema formulations.

It is envisioned that the continuous administration or sustained delivery of the compounds and compositions of the present invention may be advantageous for a given condition. While continuous administration may be accomplished via a mechanical means, such as with an infusion pump, it is contemplated that other modes of continuous or near continuous administration may be practiced. For example, such administration may be by subcutaneous or muscular injections as well as oral pills.

Techniques for formulating a variety of other sustained- or controlled-delivery means, such as liposome carriers, bio-erodible particles or beads, and depot injections, are also known to those skilled in the art.

### Dosage

Dosage levels on the order of about 0.001 mg to about 100 mg per kilogram body weight of the active ingredient compounds or compositions are useful in the treatment of the above conditions, with preferred levels ranging from 200 mg per day to 1600 mg per day. The compounds and compositions of the present invention may usually be given in two or three doses daily. Starting with a low dose (200-300 mg) twice daily and slowly working up to higher doses if needed is a preferred strategy. The amount of active ingredient that may be combined with the carrier materials to produce a single dosage form will vary depending upon the host treated and the particular mode of administration.

It is understood, however, that a specific dose level for any particular patient will depend upon a variety of factors, including the activity of the specific compound employed; the age, body weight, general health, sex, and diet of the patient; the time of administration; the rate of excretion; drug combination; the severity of the particular disorder being treated; and the form of administration. One of ordinary skill in the art would appreciate the variability of such factors and would be able to establish specific dose levels using no more than routine experimentation.

### EXAMPLES

The following examples are illustrative of the present invention and are not intended to be limitations thereon. Unless otherwise indicated, all percentages are based upon 100% by weight of the final composition.

### Example 1

### Preparation of α-(S-adenosylmethionine)-O-tocopherol

N-Acetyl-S-benzyl-L-homocysteine (0.5 g) was dissolved in 1,4-dioxane (25 ml). Dicyclohexyl carbodiimide (0.5 g) was added to the solution with stirring followed by the addition of α-tocopherol (1 g). The resulting reaction mixture was stirred at 30°-32° C. for eighteen (18) hours during which time a white precipitate separated. The mixture was then filtered and the filtrate evaporated to dryness in vacuo to give an expected oily residue.

The oily residue was added to dry ammonia (ca. 100 ml) which had been previously condensed in a 500 ml three-necked flask equipped with a stirrer and sodium hydroxide tube to maintain anhydrous conditions. While stirring, sodium was added to the reaction mixture in small pieces until the resulting blue color persisted for 5-10 minutes.

5′-O-p-Tolyl-sulfonyladenosine (0.5 g) was then added to the solution and stirring continued for ten (10) minutes. The ammonia was evaporated for three hours and the final traces thereof removed under diminished pressure, yielding a waxy solid residue. The residue was extracted with methylene chloride (2×25 ml) and the combined residue evaporated to dryness to give a waxy solid.

The waxy solid was dissolved in dimethyl sulfoxide (10 ml) containing acetic acid (3 ml) and the solution stirred with excess methyl iodide (1 ml) for 30 hours at 30°-32° C. The solvent was allowed to evaporate and the resulting residue was extracted with methylene chloride (25 ml) and dried with sodium sulfate. Evaporation of the solvent gave a clear oil which turned green when exposed to air.

The expected compound, α-(S-adenosylmethionine)-O-tocopherol was recovered and stored for future use.

### Example 2

A patient is suffering from depression. A vitamin E derivative as identified above, or a pharmaceutical composition comprising the same, may be administered to the patient. Reduction or elimination of depression and mood enhancement are expected to occur following treatment.

### Example 3

A patient is suffering from a liver disease or disorder involving hepatic glutathione levels. A vitamin E derivative as identified above, or a pharmaceutical composition comprising the same, may be administered to the patient. Enhancement of liver detoxification function is expected to occur following treatment.

### Example 4

A patient is suffering from impaired heart and/or artery function resulting from elevated blood levels of homocysteine. A vitamin E derivative as identified above, or a pharmaceutical composition comprising the same, may be administered to the patient. Enhancement of heart and/or artery function, and/or reduction in heart and/or artery disease risk is expected to occur following treatment.

### Example 5

A patient is suffering from degenerative joint disease. A vitamin E derivative as identified above, or a pharmaceutical composition comprising the same, may be administered to the patient. Stimulation of chondrocytes to increase production of new cartilage and enhancement of joint health, mobility, and comfort are expected to occur following treatment.

### Example 6

A patient is suffering from a disease or disease condition induced or exacerbated by cellular senescence. A vitamin E derivative as identified above, or a pharmaceutical composition comprising the same, may be administered to the patient. Reduction or elimination of cellular senescence is expected to occur following treatment.

### Example 7

A patient is suffering from a wound caused by physical trauma. A vitamin E derivative as identified above, or a pharmaceutical composition comprising the same, may be administered to the patient. Accelerated wound healing and/or diminished scarring are expected to occur following treatment.

### Example 8

A patient is suffering from atherosclerosis. A vitamin E derivative as identified above, or a pharmaceutical composition comprising the same, may be administered to the patient. Reduction or elimination of arterial plaques and/or reduction of oxidized low density lipoprotein are expected to occur following treatment.

### Example 9

A patient is suffering from age-related macular degeneration. A vitamin E derivative as identified above, or a pharmaceutical composition comprising the same, may be administered to the patient. Reduction or elimination of oxidative free radical damage to eye tissues is expected to occur following treatment.

### Example 10

A patient is suffering from high circulatory lipoprotein A level. A vitamin E derivative as identified above, or a pharmaceutical composition comprising the same, may be administered to the patient. Reduction of lipoprotein A level is expected to occur following treatment.

The invention being thus described, it will be obvious that the same may be varied in many ways. Such variations are not to be regarded as a departure from the spirit and scope of the invention and all such modifications are intended to be included within the scope of the following claims.

We claim:

1. A compound of formula I: 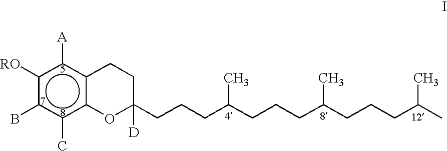
or a pharmaceutically acceptable salt, ester, or solvate, thereof, wherein:
A, B, C, and D are independently hydrogen or methyl;
R is a reaction product derived from a reactant compound selected from the group consisting of (flava-3-ol)ₙ, wherein n is 1-12, α-ketoglutaric acid, α-butyric acid, β-Carotene, 1-methylinosine, 1-methylguanosine, 1-methyladenosine, 1-methylpseudouridine, 1,3-bisphosphoglycerate, 1,3-diphosphoglycerate, 2-methyladenosine, 2-methylthio-N6-isopentenyladenosine, 2-thiouridine, 2-thiocytidine, 2′-O-methylpseudouridine, 2′-O-methyluridine, 2′-O-methylcytidine, 2′-O-methyl-N2,N2-dimethyl-guanosine, 2′-O-methyl-5-methyluridine, 2′-O-methyl-1-methyladenosine, 2-O-methylguanosine, 2′-O-methyl-N6-methyladenosine, 2′-O-methyladenosine, 2,6-di(tert-butyl)-4-methylphenol, 3-(3-amino-3-carboxypropyl)uridine, 3-carboxy-3-aminopropyl analogues, 3-methylcytidine, 3-methoxy-4-hydroxymandelic acid, 3-phosphoglycerate, 4-thiouridine, 5-carboxymethylaminomethyluridine, 5-methylaminomethyl-2-thiouridine, 5-carboxymethylaminomethyl-2-thiouridine, 5-methylcytidine, 5-methyluridine, 5-(carboxyhydroxymethyl)uridine methyl ester, 5-(carboxyhydroxymethyl)uridine, 5-carbamoylmethyluridine, 5-methyl-2-thiouridine, 5-methoxyuridine, 5-methoxycarbonylmethyl-2-thiouridine, 5-methylaminomethyluridine, 5-methyoxycarbonylmethyluridine, 5-phosphoribose-1-pyrophosphoric acid, 6-gingerol, 7-methylguanosine, acetyl-L-carnitine, acetylcholine, acyl gulycerols, ajoene, alanine, aminocyclopropane-carboxylic acid (ACC), anserine, anthocyanin, apigenin, arachidonic acid, arginine, astaxanthin, beta,D-galactosylqueuosine, beta,D-mannosylqueuosine, betaine, biopterin, biotin coenzymes such as carboxy biotin, biotin, calcium pectate, carbamyl phosphate, carnitine, carnosine, catechin, chlorogenic acid, cholesterol, choline, chondronectin, cis-zeatin, cis-aconitic acid, citric acid, Co-enzyme Q₁₀, cobamide coenzymes such as methylcobalamine or deoxyadenosylcobalamine, creatine, creatinine, cryptoxanthin, cumic acid, cumidine, curcumin, cyanidin chloride, cytochrome B, cytochrome B₅₅₈, cytochrome A, cytochrome C₁, cytochrome A₃, d-limonene, daidzein, diacylglycerol and its derivatives, dicetyl phosphate, diglycerides, dihydrouridine, dihydroxyacetone-phosphate, diphosphatidyl glycerol, diphthamide, dopamine, elastin, ellagic acid, epicatechin gallate, epicatechin, epigallocatechin, epigallocatechin gallate, epinephrine, erythrose-4-phosphate, ether phospholipids, farnesyl, fibronectins, fisetin, flavin coenzymes such as flavin mononucleotide or flavin adenine dinucleotide, flavoxanthine, folic acid, folic or pteridine coenzymes such as tetrahydrofolic acid, formic acid, fructose 1,6-bisphosphate, fructose-6-phosphate, fumaric acid, gallic acid, genistein, geranyl, ginkgolide A, ginkgolide B, ginkgolide C, glucose, glutamic acid, glutamine, glutathione, glyceraldehyde-3-phosphate, glycolipids, glycoproteins, GTP, hesperidin, histamine, histones, HMG Co-A, homoserine lactone, homoserine, indole-3-carbinol, inosinate, inosine, inositol, isocitric acid, isoleucine, kynurenine, L-histidine, L-dopa, laminin, lecithin, leucine, leukotrienes, linatine, lipoic coenzymes such as reduced or oxidized lipoamide, lipoic acid, lupeol, lutein, luteolin, lycopene, lycophyll, lycoxanthine, lysine, lysolecithin, lysophospholipids, malic acid, mandelic acid, melanins, melatonin, metanephrine, methionine, methylated estrogen, methylated lipids, methylated histones, methylated glycolipids, methylated sugars, methylated nucleic acids, methylated proteins, methylated ribosomal proteins, methylated lipids, methylated neurotransmitters, methylated glycoproteins, N-((9-beta-D-ribofuranosylpurine-6-yl)carbamoyl)-threoni ne, N-methylglycine, N-methyl histamine, N-malonyl ACC, N-((9-beta-D-ribofuranosylpurine-6-yl)n-methyl-carbamoyl) threonine, N-((9-beta-D-ribofuranosyl-2-methylthiopurine-2-yl)-carb amoyl)threonine, N-acetyl-neurominic acid, N2-(5-amino-5-carboxypentyl)cytidine, N2-methylguanosine, N2,N2-dimethlyguanosine, N4-acetylcytidine, N6-methyladenosine, N6-isopentenyladenosine, neopterin, nervonic acid, nicotinamide coenzymes such as nicotinamide adenine dinucleotide or nicotinamide adenine dinucleotide phosphate, nicotinic acid, N,N-dimethylglycine, N,N-dimethyltryptamine, norepinephrine, normetanephrine, nucleotide coenzymes such as UDP-glucose, CDP-choline, CDP-diacylglycerol, CMP-sialic acid, and other nucleotide derivatives of carbohydrates, alcohols, amino acids, lipids, or inorganic compounds, Omega-3 fatty acids, ornithine, oxaloacetic acid, p-coumaric acid, palmitic acid, pantothenic coenzymes such as pantothenic CoA, pantothenic dephospho-CoA, or 4-phosphopantothenate, pantothenic acid, para-aminobenzoic acid (PABA), pectin, phenylalanine, phosphatidyl ethanolamine, phosphatidyl choline, phosphocreatine, phosphoenol pyruvate, phosphonolipids, phytic acid, phytochlorin, phytol, picolinic acid, plasmalogens, plastoquinone, proanthocyanin, pseudouridine, pyridoxine coenzymes such as pyridoxal-phosphate or pyridoxamine-phosphate, pyruvate, pyruvate, quercetin, queuine, queuosine, quinolinic acid, ribose-5-phosphate, ribosomal proteins, ribulose-1,5-biphosphate, rutin, S-allymercaptocysteine, sarcosine, sedoheptulose-1,7-bisphosphate, sedoheptulose-7-phosphate, serotonin, sesamin, silybin, soy isoflavonoids, sphingolipids, sphingomyelin, sphingosine, steraylamine, succinic acid, sugars such as lactose, glucose, or mannose, sulforaphone, sulphorane, taurine, taxicatin, taxicin I, taxicin II, taxifolin, taxine A, taxodione, testosterone, tetrahydrobiopterin and its derivatives, tetrahydrofolate, thiamine coenzymes such as thiamine monophosphate, thiamine diphosphate, or thiamine triphosphate, threonine, trimethylysine, tryptamine, tryptophane, tumeric, tyrosine, uridine-5-oxyacetic acid, uridine-5-oxoacetic acid methyl ester, vaccenic acid, valine, vanillic acid, vitamin A, vitamin B₁ (Thiamine), vitamin B₂ (Riboflavin), vitamin B₆ (Pyrodoxine), vitamin B₁₂ (Cyanocobalamine), vitamin C (esterified or non-esterified Ascorbic Acid), vitamin D (Ergocalciferol), wybutosine, wybutoxosine, wye base, xanthophyll, xanthoxylin, xylulose-5-phosphate, and zeaxanthin;
R is a straight or branched C₁-C₃₀ alkyl, or C₂-C₃₀ straight or branched alkenyl or alkynyl optionally substituted with 1 to 12 substituents selected from the group consisting of hydroxy, carboxy, amino, halo, nitro, sulfhydryl, and J, wherein J is phenyl or a 5-7 membered heterocyclic ring which has one or more O, N, or S as the heteroatom(s), and J is optionally substituted with 1 to 5 substituents selected from the group consisting of hydroxy, carboxy, amino, halo, nitro, sulfhydryl, methyl, straight or branched C₂-C₁₀ alkyl, alkenyl, or alkynyl, methoxy, C₂-C₈ straight or branched alkoxy, and —OC(O)R₂ where R₂ is trifluoromethyl, methyl, straight or branched C₁-C₁₀ alkyl, or straight or branched C₂-C₁₀ alkenyl or alkynyl, wherein said hydroxy, carboxy, amino, halo, nitro, sulfhydryl, C₁-C₁₀ alkyl, C₂-C₁₀ alkenyl or alkynyl, methoxy, C₂-C₈ straight or branched alkoxy, and —OC(O)R₂ substituents are optionally substituted; and wherein one or more carbon atom(s) of said alkyl, alkenyl, alkynyl, or alkoxy, is optionally replaced with nitrogen, oxygen, or sulfur; or
R is phenyl optionally substituted with 1 to 5 substituents selected from the group consisting of hydroxy, carboxy, amino, halo, nitro, sulfhydryl, trifluoromethyl, methyl, C₁-C₈ straight or branched alkyl, C₂-C₈ straight or branched alkenyl or alkynyl, methoxy, and C₂-C₈ straight or branched alkoxy, wherein said hydroxy, carboxy, amino, sulfhydryl, halo, nitro, C₁-C₈ straight or branched alkyl, C₂-C₈ straight or branched alkenyl or alkynyl, methoxy, and C₂-C₈ straight or branched alkoxy substituents are optionally substituted; and wherein one or more carbon atom(s) of said alkyl, alkenyl, alkynyl, or alkoxy, is optionally replaced with nitrogen, oxygen, or sulfur; and
and wherein the stereochemistry at each of the 2, 4′, and 8′ positions is R or S.

2. The compound of claim 1, wherein D is methyl.

3. The compound of claim 2, wherein A, B, and C are each methyl.

4. The compound of claim 2, wherein A and C are each methyl, and B is hydrogen.

5. The compound of claim 2, wherein A is hydrogen, and B and C are each methyl.

6. The compound of claim 2, wherein A and B are each hydrogen, and C is methyl.

7. A pharmaceutical composition comprising:
(i) an effective amount of the compound of formula I: 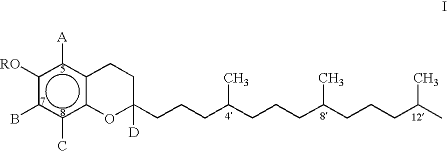
or a pharmaceutically acceptable salt, ester, or solvate, thereof, wherein:
A, B, C, and D are independently hydrogen or methyl;
R is a reaction product derived from a reactant compound selected from the group consisting of (flava-3-ol)ₙ, wherein n is 1-12, α-ketoglutaric acid, α-butyric acid, β-Carotene, 1-methylinosine, 1-methylguanosine , 1-methyladenosine, 1-methylpseudouridine, 1,3-bisphosphoglycerate, 1,3-diphosphoglycerate, 2-methyladenosine, 2-methylthio-N6-isopentenyladenosine, 2-thiouridine, 2-thiocytidine, 2′-O-methylpseudouridine, 2′-O-methyluridine, 2′-O-methylcytidine, 2′-O-methyl-N2,N2-dimethyl-guanosine, 2′-O-methyl-5-methyluridine, 2′-O-methyl-1-methyladenosine, 2′-O-methylguanosine, 2′-O-methyl-N6-methyladenosine, 2′-O-methyladenosine, 2,6-di(tert-butyl)-4-methylphenol, 3-(3-amino-3-carboxypropyl)uridine, 3-carboxy-3-aminopropyl analogues, 3-methylcytidine, 3-methoxy-4-hydroxymandelic acid, 3-phosphoglycerate, 4-thiouridine, 5-carboxymethylaminomethyluridine, 5-methylaminomethyl-2-thiouridine, 5-carboxymethylaminomethyl-2-thiouridine, 5-methylcytidine, 5-methyluridine, 5-(carboxyhydroxymethyl)uridine methyl ester, 5-(carboxyhydroxymethyl)uridine, 5-carbamoylmethyluridine, 5-methyl-2-thiouridine, 5-methoxyuridine, 5-methoxycarbonylmethyl-2-thiouridine, 5-methylaminomethyluridine, 5-methyoxycarbonylmethyluridine, 5-phosphoribose-1-pyrophosphoric acid, 6-gingerol, 7-methylguanosine, acetyl-L-carnitine, acetylcholine, acyl gulycerols, ajoene, alanine, aminocyclopropane-carboxylic acid (ACC), anserine, anthocyanin, apigenin, arachidonic acid, arginine, astaxanthin, beta,D-galactosylqueuosine, beta,D-mannosylqueuosine, betaine, biopterin, biotin coenzymes such as carboxy biotin, biotin, calcium pectate, carbamyl phosphate, carnitine, carnosine, catechin, chlorogenic acid, cholesterol, choline, chondronectin, cis-zeatin, cis-aconitic acid, citric acid, Co-enzyme Q₁₀, cobamide coenzymes such as methylcobalamine or deoxyadenosylcobalamine, creatine, creatinine, cryptoxanthin, cumic acid, cumidine, curcumin, cyanidin chloride, cytochrome B, cytochrome B₅₅₈, cytochrome A, cytochrome C₁, cytochrome A₃, d-limonene, daidzein, diacylglycerol and its derivatives, dicetyl phosphate, diglycerides, dihydrouridine, dihydroxyacetone-phosphate, diphosphatidyl glycerol, diphthamide, dopamine, elastin, ellagic acid, epicatechin gallate, epicatechin, epigallocatechin, epigallocatechin gallate, epinephrine, erythrose-4-phosphate, ether phospholipids, farnesyl, fibronectins, fisetin, flavin coenzymes such as flavin mononucleotide or flavin adenine dinucleotide, flavoxanthine, folic acid, folic or pteridine coenzymes such as tetrahydrofolic acid, formic acid, fructose 1,6-bisphosphate, fructose-6-phosphate, fumaric acid, gallic acid, genistein, geranyl, ginkgolide A, ginkgolide B, ginkgolide C, glucose, glutamic acid, glutamine, glutathione, glyceraldehyde-3-phosphate, glycolipids, glycoproteins, GTP, hesperidin, histamine, histones, HMG Co-A, homoserine lactone, homoserine, indole-3-carbinol, inosinate, inosine, inositol, isocitric acid, isoleucine, kynurenine, L-histidine, L-dopa, laminin, lecithin, leucine, leukotrienes, linatine, lipoic coenzymes such as reduced or oxidized lipoamide, lipoic acid, lupeol, lutein, luteolin, lycopene, lycophyll, lycoxanthine, lysine, lysolecithin, lysophospholipids, malic acid, mandelic acid, melanins, melatonin, metanephrine, methionine, methylated estrogen, methylated lipids, methylated histones, methylated glycolipids, methylated sugars, methylated nucleic acids, methylated proteins, methylated ribosomal proteins, methylated lipids, methylated neurotransmitters, methylated glycoproteins, N-((9-beta-D-ribofuranosylpurine-6-yl)carbamoyl)-threoni ne, N-methylglycine, N-methyl histamine, N-malonyl ACC, N-((9-beta-D-ribofuranosylpurine-6-yl)n-methyl-carbamoyl) threonine, N-((9-beta-D-ribofuranosyl-2-methylthiopurine-2-yl)-carb amoyl)threonine, N-acetyl-neurominic acid, N2-(5-amino-5-carboxypentyl)cytidine, N2-methylguanosine, N2,N2-dimethlyguanosine, N4-acetylcytidine, N6-methyladenosine, N6-isopentenyladenosine, neopterin, nervonic acid, nicotinamide coenzymes such as nicotinamide adenine dinucleotide or nicotinamide adenine dinucleotide phosphate, nicotinic acid, N,N-dimethylglycine, N,N-dimethyltryptamine, norepinephrine, normetanephrine, nucleotide coenzymes such as UDP-glucose, CDP-choline, CDP-diacylglycerol, CMP-sialic acid, and other nucleotide derivatives of carbohydrates, alcohols, amino acids, lipids, or inorganic compounds, Omega-3 fatty acids, ornithine, oxaloacetic acid, p-coumaric acid, palmitic acid, pantothenic coenzymes such as pantothenic CoA, pantothenic dephospho-CoA, or 4-phosphopantothenate, pantothenic acid, para-aminobenzoic acid (PABA), pectin, phenylalanine, phosphatidyl ethanolamine, phosphatidyl choline, phosphocreatine, phosphoenol pyruvate, phosphonolipids, phytic acid, phytochlorin, phytol, picolinic acid, plasmalogens, plastoquinone, proanthocyanin, pseudouridine, pyridoxine coenzymes such as pyridoxal-phosphate or pyridoxamine-phosphate, pyruvate, pyruvate, quercetin, queuine, queuosine, quinolinic acid, ribose-5-phosphate, ribosomal proteins, ribulose-1,5-biphosphate, rutin, S-allymercaptocysteine, sarcosine, sedoheptulose-1,7-bisphosphate, sedoheptulose-7-phosphate, serotonin, sesamin, silybin, soy isoflavonoids, sphingolipids, sphingomyelin, sphingosine, steraylamine, succinic acid, sugars such as lactose, glucose, or mannose, sulforaphone, sulphorane, taurine, taxicatin, taxicin I, taxicin II, taxifolin, taxine A, taxodione, testosterone, tetrahydrobiopterin and its derivatives, tetrahydrofolate, thiamine coenzymes such as thiamine monophosphate, thiamine diphosphate, or thiamine triphosphate, threonine, trimethylysine, tryptamine, tryptophane, tumeric, tyrosine, uridine-5-oxyacetic acid, uridine-5-oxoacetic acid methyl ester, vaccenic acid, valine, vanillic acid, vitamin A, vitamin B₁ (Thiamine), vitamin B₂ (Riboflavin), vitamin B₆ (Pyrodoxine), vitamin B₁₂ (Cyanocobalamine), vitamin C (esterified or non-esterified Ascorbic Acid), vitamin D (Ergocalciferol), wybutosine, wybutoxosine, wye base, xanthophyll, xanthoxylin, xylulose-5-phosphate, and zeaxanthin;
R is a straight or branched C₁-C₃₀ alkyl, or C₂-C₃₀ straight or branched alkenyl or alkynyl optionally substituted with 1 to 12 substituents selected from the group consisting of hydroxy, carboxy, amino, halo, nitro, sulfhydryl, and J, wherein J is phenyl or a 5-7 membered heterocyclic ring which has one or more O, N, or S as the heteroatom(s), and J is optionally substituted with 1 to 5 substituents selected from the group consisting of hydroxy, carboxy, amino, halo, nitro, sulfhydryl, methyl, straight or branched C₂-C₁₀ alkyl, alkenyl, or alkynyl, methoxy, C₂-C₈ straight or branched alkoxy, and —OC(O)R₂ where R₂ is trifluoromethyl, methyl, straight or branched C₁-C₁₀ alkyl, or straight or branched C₂-C₁₀ alkenyl or alkynyl, wherein said hydroxy, carboxy, amino, halo, nitro, sulfhydryl, C₁-C₁₀ alkyl, C₂-C₁₀ alkenyl or alkynyl, methoxy, C₂-C₈ straight or branched alkoxy, and —OC(O)R₂ substituents are optionally substituted; and wherein one or more carbon atom(s) of said alkyl, alkenyl, alkynyl, or alkoxy, is optionally replaced with nitrogen, oxygen, or sulfur; or
R is phenyl optionally substituted with 1 to 5 substituents selected from the group consisting of hydroxy, carboxy, amino, halo, nitro, sulfhydryl, trifluoromethyl, methyl, C₁-C₈ straight or branched alkyl, C₂-C₈ straight or branched alkenyl or alkynyl, methoxy, and C₂-C₈ straight or branched alkoxy, wherein said hydroxy, carboxy, amino, sulfhydryl, halo, nitro, C₁-C₈ straight or branched alkyl, C₂-C₈ straight or branched alkenyl or alkynyl, methoxy, and C₂-C₈ straight or branched alkoxy substituents are optionally substituted; and wherein one or more carbon atom(s) of said alkyl, alkenyl, alkynyl, or alkoxy, is optionally replaced with nitrogen, oxygen, or sulfur;
wherein the stereochemistry at each of the 2, 4′, and 8′ positions is R or S; and
(ii) a pharmaceutically acceptable carrier.

8. A method for effecting a biological activity in an animal, which comprises administering to said animal an effective amount of a compound of formula I of claim 1, wherein the biological activity is selected from the group consisting of aging or longevity, nerve activity, hematopoiesis or maintenance of blood cells, hepatic activity, nephritic activity, heart and cardiovascular function, pulmonary function, muscular function, cartilage, bone, and joint health, gastrointestinal function, reproductive system function, vision, immune function, cell membrane integrity, and pain and inflammation; preventing or treating diseases or conditions; treating cancers or obesity; and reducing the risk of Sudden Infant Death Syndrome.

9. The method of claim 8, wherein said effect on neurochemical activity is selected from the group consisting of treating anxiety; treating depression; treating depression secondary to a chronic disease; treating dementia; treating schizophrenia; treating Alzheimer's disease; treating Parkinson's disease; treating demyelinating disorders; treating peripheral neuropathies; enhancing mood and behavior; and maintaining or effecting neuronal membrane ratios of phosphatidyl choline and cholesterol.

10. The method of claim 8, wherein said effect on liver biology activity is selected from the group consisting of treating cirrhosis, chronic liver disease, alcoholic liver damage, toxic chemical exposure, NSAID-liver damage, estrogen induced liver problems, bile disorders, and environmental chemical hypersensitivity.

11. The method of claim 8, wherein said effect on heart and artery function is treating or reducing heart and/or artery disease risk due to elevated blood levels of homocysteine.

12. The method of claim 8, wherein said effect on cartilage, bone and joint health is selected from the group consisting of treating osteoarthritis, rheumatoid arthritis, fibromyalgia, joint injuries, joint inflammation, joint degeneration, and osteoporosis.

13. The method of claim 8, wherein said effect on immune function is selected from the group consisting of treating organ transplant rejection, graft rejection, lupus, uveitis, Behcet's disease, Graves disease, Guillain-Barre syndrome, psoriasis, acute dermatomyositis, atopic skin disease, scleroderma, eczema, aplastic anemia, primary cirrhosis, autoimmune hepatitis, ulcerative colitis, Crohn's disease, amyotrophic lateral sclerosis, myasthenia gravis, multiple sclerosis, hepatic syndrome, glomerulonephritis, rheumatoid arthritis, and diabetes mellitus.

14. A compound of formula II 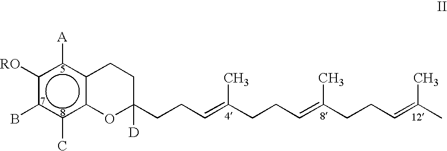
or a pharmaceutically acceptable salt, ester, or solvate thereof, wherein:
A, B, C, and D are independently hydrogen or methyl;
R is a reaction product derived from a reactant compound selected from the group consisting of (flava-3-ol)ₙ, wherein n is 1-12, α-ketoglutaric acid, α-butyric acid, β-Carotene, 1-methylinosine, 1-methylguanosine, 1-methyladenosine, 1-methylpseudouridine, 1,3-bisphosphoglycerate, 1,3-diphosphoglycerate, 2-methyladenosine, 2-methylthio-N6-isopentenyladenosine, 2-thiouridine, 2-thiocytidine, 2′-O-methylpseudouridine, 2′-O-methyluridine, 2′-O-methylcytidine, 2′-O-methyl-N2,N2-dimethyl-guanosine, 2′-O-methyl-5-methyluridine, 2′-O-methyl-1-methyladenosine, 2′-O-methylguanosine, 2′-O-methyl-N6-methyladenosine, 2′-O-methyladenosine, 2,6-di(tert-butyl)-4-methylphenol, 3-(3-amino-3-carboxypropyl)uridine, 3-carboxy-3-aminopropyl analogues, 3-methylcytidine, 3-methoxy-4-hydroxymandelic acid, 3-phosphoglycerate, 4-thiouridine, 5-carboxymethylaminomethyluridine, 5-methylaminomethyl-2-thiouridine, 5-carboxymethylaminomethyl-2-thiouridine, 5-methylcytidine, 5-methyluridine, 5-(carboxyhydroxymethyl)uridine methyl ester, 5-(carboxyhydroxymethyl)uridine, 5-carbamoylmethyluridine, 5-methyl-2-thiouridine, 5-methoxyuridine, 5-methoxycarbonylmethyl-2-thiouridine, 5-methylaminomethyluridine, 5-methyoxycarbonylmethyluridine, 5-phosphoribose-1-pyrophosphoric acid, 6-gingerol, 7-methylguanosine, acetyl-L-carnitine, acetylcholine, acyl gulycerols, ajoene, alanine, aminocyclopropane-carboxylic acid (ACC), anserine, anthocyanin, apigenin, arachidonic acid, arginine, astaxanthin, beta,D-galactosylqueuosine, beta,D-mannosylqueuosine, betaine, biopterin, biotin coenzymes such as carboxy biotin, biotin, calcium pectate, carbamyl phosphate, carnitine, carnosine, catechin, chlorogenic acid, cholesterol, choline, chondronectin, cis-zeatin, cis-aconitic acid, citric acid, Co-enzyme Q₁₀, cobamide coenzymes such as methylcobalamine or deoxyadenosylcobalamine, creatine, creatinine, cryptoxanthin, cumic acid, cumidine, curcumin, cyanidin chloride, cytochrome B, cytochrome B₅₅₈, cytochrome A, cytochrome C₁, cytochrome A₃, d-limonene, daidzein, diacylglycerol and its derivatives, dicetyl phosphate, diglycerides, dihydrouridine, dihydroxyacetone-phosphate, diphosphatidyl glycerol, diphthamide, dopamine, elastin, ellagic acid, epicatechin gallate, epicatechin, epigallocatechin, epigallocatechin gallate, epinephrine, erythrose-4-phosphate, ether phospholipids, farnesyl, fibronectins, fisetin, flavin coenzymes such as flavin mononucleotide or flavin adenine dinucleotide, flavoxanthine, folic acid, folic or pteridine coenzymes such as tetrahydrofolic acid, formic acid, fructose 1,6-bisphosphate, fructose-6-phosphate, fumaric acid, gallic acid, genistein, geranyl, ginkgolide A, ginkgolide B, ginkgolide C, glucose, glutamic acid, glutamine, glutathione, glyceraldehyde-3-phosphate, glycolipids, glycoproteins, GTP, hesperidin, histamine, histones, HMG Co-A, homoserine lactone, homoserine, indole-3-carbinol, inosinate, inosine, inositol, isocitric acid, isoleucine, kynurenine, L-histidine, L-dopa, laminin, lecithin, leucine, leukotrienes, linatine, lipoic coenzymes such as reduced or oxidized lipoamide, lipoic acid, lupeol, lutein, luteolin, lycopene, lycophyll, lycoxanthine, lysine, lysolecithin, lysophospholipids, malic acid, mandelic acid, melanins, melatonin, metanephrine, methionine, methylated estrogen, methylated lipids, methylated histones, methylated glycolipids, methylated sugars, methylated nucleic acids, methylated proteins, methylated ribosomal proteins, methylated lipids, methylated neurotransmitters, methylated glycoproteins, N-((9-beta-D-ribofuranosylpurine-6-yl)carbamoyl)-threoni ne, N-methylglycine, N-methyl histamine, N-malonyl ACC, N-((9-beta-D-ribofuranosylpurine-6-yl)n-methyl-carbamoyl) threonine, N-((9-beta-D-ribofuranosyl-2-methylthiopurine-2-yl)-carb amoyl)threonine, N-acetyl-neurominic acid, N2-(5-amino-5-carboxypentyl)cytidine, N2-methylguanosine, N2,N2-dimethlyguanosine, N4-acetylcytidine, N6-methyladenosine, N6-isopentenyladenosine, neopterin, nervonic acid, nicotinamide coenzymes such as nicotinamide adenine dinucleotide or nicotinamide adenine dinucleotide phosphate, nicotinic acid, N,N-dimethylglycine, N,N-dimethyltryptamine, norepinephrine, normetanephrine, nucleotide coenzymes such as UDP-glucose, CDP-choline, CDP-diacylglycerol, CMP-sialic acid, and other nucleotide derivatives of carbohydrates, alcohols, amino acids, lipids, or inorganic compounds, Omega-3 fatty acids, ornithine, oxaloacetic acid, p-coumaric acid, palmitic acid, pantothenic coenzymes such as pantothenic CoA, pantothenic dephospho-CoA, or 4-phosphopantothenate, pantothenic acid, para-aminobenzoic acid (PABA), pectin, phenylalanine, phosphatidyl ethanolamine, phosphatidyl choline, phosphocreatine, phosphoenol pyruvate, phosphonolipids, phytic acid, phytochlorin, phytol, picolinic acid, plasmalogens, plastoquinone, proanthocyanin, pseudouridine, pyridoxine coenzymes such as pyridoxal-phosphate or pyridoxamine-phosphate, pyruvate, pyruvate, quercetin, queuine, queuosine, quinolinic acid, ribose-5-phosphate, ribosomal proteins, ribulose-1,5-biphosphate, rutin, S-allymercaptocysteine, sarcosine, sedoheptulose-1,7-bisphosphate, sedoheptulose-7-phosphate, serotonin, sesamin, silybin, soy isoflavonoids, sphingolipids, sphingomyelin, sphingosine, steraylamine, succinic acid, sugars such as lactose, glucose, or mannose, sulforaphone, sulphorane, taurine, taxicatin, taxicin I, taxicin II, taxifolin, taxine A, taxodione, testosterone, tetrahydrobiopterin and its derivatives, tetrahydrofolate, thiamine coenzymes such as thiamine monophosphate, thiamine diphosphate, or thiamine triphosphate, threonine, trimethylysine, tryptamine, tryptophane, tumeric, tyrosine, uridine-5-oxyacetic acid, uridine-5-oxoacetic acid methyl ester, vaccenic acid, valine, vanillic acid, vitamin A, vitamin B₁ (Thiamine), vitamin B₂ (Riboflavin), vitamin B₆ (Pyrodoxine), vitamin B₁₂ (Cyanocobalamine), vitamin C (esterified or non-esterified Ascorbic Acid), vitamin D (Ergocalciferol), wybutosine, wybutoxosine, wye base, xanthophyll, xanthoxylin, xylulose-5-phosphate, and zeaxanthin;
R is a straight or branched C₁-C₃₀ alkyl, or C₂-C₃₀ straight or branched alkenyl or alkynyl optionally substituted with 1 to 12 substituents selected from the group consisting of hydroxy, carboxy, amino, halo, nitro, sulfhydryl, and J, wherein J is phenyl or a 5-7 membered heterocyclic ring which has one or more O, N, or S as the heteroatom(s), and J is optionally substituted with 1 to 5 substituents selected from the group consisting of hydroxy, carboxy, amino, halo, nitro, sulfhydryl, methyl, straight or branched C₂-C₁₀ alkyl, alkenyl, or alkynyl, methoxy, C₂-C₈ straight or branched alkoxy, and —OC(O)R₂ where R₂ is trifluoromethyl, methyl, straight or branched C₁-C₁₀ alkyl, or straight or branched C₂-C₁₀ alkenyl or alkynyl, wherein said hydroxy, carboxy, amino, halo, nitro, sulfhydryl, C₁-C₁₀ alkyl, C₂-C₁₀ alkenyl or alkynyl, methoxy, C₂-C₈ straight or branched alkoxy, and —OC(O)R₂ substituents are optionally substituted; and wherein one or more carbon atom(s) of said alkyl, alkenyl, alkynyl, or alkoxy, is optionally replaced with nitrogen, oxygen, or sulfur; or
R is phenyl optionally substituted with 1 to 5 substituents selected from the group consisting of hydroxy, carboxy, amino, halo, nitro, sulfhydryl, trifluoromethyl, methyl, C₁-C₈ straight or branched alkyl, C₂-C₈ straight or branched alkenyl or alkynyl, methoxy, and C₂-C₈ straight or branched alkoxy, wherein said hydroxy, carboxy, amino, sulfhydryl, halo, nitro, C₁-C₈ straight or branched alkyl, C₂-C₈ straight or branched alkenyl or alkynyl, methoxy, and C₂-C₈ straight or branched alkoxy substituents are optionally substituted; and wherein one or more carbon atom(s) of said alkyl, alkenyl, alkynyl, or alkoxy, is optionally replaced with nitrogen, oxygen, or sulfur;
and wherein the stereochemistry at each of the 2, 4′, and 8′ positions is R or S.

15. The compound of claim 14, wherein D is methyl.

16. The compound of claim 15, wherein A, B, and C are each methyl.

17. The compound of claim 16, wherein A and C are each methyl, and B is hydrogen.

18. The compound of claim 17, wherein A is hydrogen, and B and C are each methyl.

19. The compound of claim 18, wherein A and B are each hydrogen, and C is methyl.

20. A pharmaceutical composition comprising:
(i) an effective amount of the compound of formula II 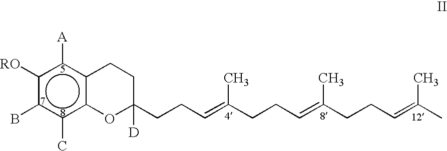
or a pharmaceutically acceptable salt, ester, or solvate thereof, wherein:
A, B, C, and D are independently hydrogen or methyl;
R is a reaction product derived from a reactant compound selected from the group consisting of (flava-3-ol)ₙ, wherein n is 1-12, α-ketoglutaric acid, α-butyric acid, β-Carotene, 1-methylinosine, 1-methylguanosine, 1-methyladenosine, 1-methylpseudouridine, 1,3-bisphosphoglycerate, 1,3-diphosphoglycerate, 2-methyladenosine, 2-methylthio-N6-isopentenyladenosine, 2-thiouridine, 2-thiocytidine, 2′-O-methylpseudouridine, 2′-O-methyluridine, 2′-O-methylcytidine, 2′-O-methyl-N2,N2-dimethyl-guanosine, 2′-O-methyl-5-methyluridine, 2′-O-methyl-1-methyladenosine, 2′-O-methylguanosine, 2′-O-methyl-N6-methyladenosine, 2′-O-methyladenosine, 2,6-di(tert-butyl)-4-methylphenol, 3-(3-amino-3-carboxypropyl)uridine, 3-carboxy-3-aminopropyl analogues, 3-methylcytidine, 3-methoxy-4-hydroxymandelic acid, 3-phosphoglycerate, 4-thiouridine, 5-carboxymethylaminomethyluridine, 5-methylaminomethyl-2-thiouridine, 5-carboxymethylaminomethyl-2-thiouridine, 5-methylcytidine, 5-methyluridine, 5-(carboxyhydroxymethyl)uridine methyl ester, 5-(carboxyhydroxymethyl)uridine, 5-carbamoylmethyluridine, 5-methyl-2-thiouridine, 5-methoxyuridine, 5-methoxycarbonylmethyl -2-thiouridine, 5-methylaminomethyluridine, 5-methyoxycarbonylmethyluridine, 5-phosphoribose-1-pyrophosphoric acid, 6-gingerol, 7-methylguanosine, acetyl-L-carnitine, acetylcholine, acyl gulycerols, ajoene, alanine, aminocyclopropane-carboxylic acid (ACC), anserine, anthocyanin, apigenin, arachidonic acid, arginine, astaxanthin, beta,D-galactosylqueuosine, beta,D-mannosylqueuosine, betaine, biopterin, biotin coenzymes such as carboxy biotin, biotin, calcium pectate, carbamyl phosphate, carnitine, carnosine, catechin, chlorogenic acid, cholesterol, choline, chondronectin, cis-zeatin, cis-aconitic acid, citric acid, Co-enzyme Q₁₀, cobamide coenzymes such as methylcobalamine or deoxyadenosylcobalamine, creatine, creatinine, cryptoxanthin, cumic acid, cumidine, curcumin, cyanidin chloride, cytochrome B, cytochrome B₅₅₈, cytochrome A, cytochrome C₁, cytochrome A₃, d-limonene, daidzein, diacylglycerol and its derivatives, dicetyl phosphate, diglycerides, dihydrouridine, dihydroxyacetone-phosphate, diphosphatidyl glycerol, diphthamide, dopamine, elastin, ellagic acid, epicatechin gallate, epicatechin, epigallocatechin, epigallocatechin gallate, epinephrine, erythrose-4-phosphate, ether phospholipids, farnesyl, fibronectins, fisetin, flavin coenzymes such as flavin mononucleotide or flavin adenine dinucleotide, flavoxanthine, folic acid, folic or pteridine coenzymes such as tetrahydrofolic acid, formic acid, fructose 1,6-bisphosphate, fructose-6-phosphate, fumaric acid, gallic acid, genistein, geranyl, ginkgolide A, ginkgolide B, ginkgolide C, glucose, glutamic acid, glutamine, glutathione, glyceraldehyde-3-phosphate, glycolipids, glycoproteins, GTP, hesperidin, histamine, histones, HMG Co-A, homoserine lactone, homoserine, indole-3-carbinol, inosinate, inosine, inositol, isocitric acid, isoleucine, kynurenine, L-histidine, L-dopa, laminin, lecithin, leucine, leukotrienes, linatine, lipoic coenzymes such as reduced or oxidized lipoamide, lipoic acid, lupeol, lutein, luteolin, lycopene, lycophyll, lycoxanthine, lysine, lysolecithin, lysophospholipids, malic acid, mandelic acid, melanins, melatonin, metanephrine, methionine, methylated estrogen, methylated lipids, methylated histones, methylated glycolipids, methylated sugars, methylated nucleic acids, methylated proteins, methylated ribosomal proteins, methylated lipids, methylated neurotransmitters, methylated glycoproteins, N-((9-beta-D-ribofuranosylpurine-6-yl)carbamoyl)-threoni ne, N-methylglycine, N-methyl histamine, N-malonyl ACC, N-((9-beta-D-ribofuranosylpurine-6-yl)n-methyl-carbamoyl) threonine, N-((9-beta-D-ribofuranosyl-2-methylthiopurine-2-yl)-carb amoyl)threonine, N-acetyl-neurominic acid, N2-(5-amino-5-carboxypentyl)cytidine, N2-methylguanosine, N2,N2-dimethlyguanosine, N4-acetylcytidine, N6-methyladenosine, N6-isopentenyladenosine, neopterin, nervonic acid, nicotinamide coenzymes such as nicotinamide adenine dinucleotide or nicotinamide adenine dinucleotide phosphate, nicotinic acid, N,N-dimethylglycine, N,N-dimethyltryptamine, norepinephrine, normetanephrine, nucleotide coenzymes such as UDP-glucose, CDP-choline, CDP-diacylglycerol, CMP-sialic acid, and other nucleotide derivatives of carbohydrates, alcohols, amino acids, lipids, or inorganic compounds, Omega-3 fatty acids, ornithine, oxaloacetic acid, p-coumaric acid, palmitic acid, pantothenic coenzymes such as pantothenic CoA, pantothenic dephospho-CoA, or 4-phosphopantothenate, pantothenic acid, para-aminobenzoic acid (PABA), pectin, phenylalanine, phosphatidyl ethanolamine, phosphatidyl choline, phosphocreatine, phosphoenol pyruvate, phosphonolipids, phytic acid, phytochlorin, phytol, picolinic acid, plasmalogens, plastoquinone, proanthocyanin, pseudouridine, pyridoxine coenzymes such as pyridoxal-phosphate or pyridoxamine-phosphate, pyruvate, pyruvate, quercetin, queuine, queuosine, quinolinic acid, ribose-5-phosphate, ribosomal proteins, ribulose-1,5-biphosphate, rutin, S-allymercaptocysteine, sarcosine, sedoheptulose-1,7-bisphosphate, sedoheptulose-7-phosphate, serotonin, sesamin, silybin, soy isoflavonoids, sphingolipids, sphingomyelin, sphingosine, steraylamine, succinic acid, sugars such as lactose, glucose, or mannose, sulforaphone, sulphorane, taurine, taxicatin, taxicin I, taxicin II, taxifolin, taxine A, taxodione, testosterone, tetrahydrobiopterin and its derivatives, tetrahydrofolate, thiamine coenzymes such as thiamine monophosphate, thiamine diphosphate, or thiamine triphosphate, threonine, trimethylysine, tryptamine, tryptophane, tumeric, tyrosine, uridine-5-oxyacetic acid, uridine-5-oxoacetic acid methyl ester, vaccenic acid, valine, vanillic acid, vitamin A, vitamin B₁ (Thiamine), vitamin B₂ (Riboflavin), vitamin B₆ (Pyrodoxine), vitamin B₁₂ (Cyanocobalamine), vitamin C (esterified or non-esterified Ascorbic Acid), vitamin D (Ergocalciferol), wybutosine, wybutoxosine, wye base, xanthophyll, xanthoxylin, xylulose-5-phosphate, and zeaxanthin;
R is a straight or branched C₁-C₃₀ alkyl, or C₂-C₃₀ straight or branched alkenyl or alkynyl optionally substituted with 1 to 12 substituents selected from the group consisting of hydroxy, carboxy, amino, halo, nitro, sulfhydryl, and J, wherein J is phenyl or a 5-7 membered heterocyclic ring which has one or more O, N, or S as the heteroatom(s), and J is optionally substituted with 1 to 5 substituents selected from the group consisting of hydroxy, carboxy, amino, halo, nitro, sulfhydryl, methyl, straight or branched C₂-C₁₀ alkyl, alkenyl, or alkynyl, methoxy, C₂-C₈ straight or branched alkoxy, and —OC(O)R₂ where R₂ is trifluoromethyl, methyl, straight or branched C₁-C₁₀ alkyl, or straight or branched C₂-C₁₀-alkenyl or alkynyl, wherein said hydroxy, carboxy, amino, halo, nitro, sulfhydryl, C₁-C₁₀ alkyl, C₂-C₁₀ alkenyl or alkynyl, methoxy, C₂-C₈ straight or branched alkoxy, and —OC(O)R₂ substituents are optionally substituted; and wherein one or more carbon atom(s) of said alkyl, alkenyl, alkynyl, or alkoxy, is optionally replaced with nitrogen, oxygen, or sulfur; or
R is phenyl optionally substituted with 1 to 5 substituents selected from the group consisting of hydroxy, carboxy, amino, halo, nitro, sulfhydryl, trifluoromethyl, methyl, C₁-C₈ straight or branched alkyl, C₂-C₈ straight or branched alkenyl or alkynyl, methoxy, and C₂-C₈ straight or branched alkoxy, wherein said hydroxy, carboxy, amino, sulfhydryl, halo, nitro, C₁-C₈ straight or branched alkyl, C₂-C₈ straight or branched alkenyl or alkynyl, methoxy, and C₂-C₈ straight or branched alkoxy substituents are optionally substituted; and wherein one or more carbon atom(s) of said alkyl, alkenyl, alkynyl, or alkoxy, is optionally replaced with nitrogen, oxygen, or sulfur;
wherein the stereochemistry at each of the 2, 4′, and 8′ positions is R or S; and
(ii) a pharmaceutically acceptable carrier.

21. A method for effecting a biological activity in an animal, which comprises administering to said animal an effective amount of a compound of formula II of claim 14, wherein the biological activity is selected from the group consisting of aging or longevity, nerve activity, hematopoiesis or maintenance of blood cells, hepatic activity, nephritic activity, heart and cardiovascular function, pulmonary function, muscular function, cartilage, bone, and joint health, gastrointestinal function, reproductive system function, vision, immune function, cell membrane integrity, and pain and inflammation; preventing or treating diseases or conditions; treating cancers or obesity; and reducing the risk of Sudden Infant Death Syndrome.

22. The method of claim 21, wherein said effect on neurochemical activity is selected from the group consisting of treating anxiety; treating depression; treating depression secondary to a chronic disease; treating dementia; treating schizophrenia; treating Alzheimer's disease; treating Parkinson's disease; treating demyelinating disorders; treating peripheral neuropathies; enhancing mood and behavior; and maintaining or effecting neuronal membrane ratios of phosphatidyl choline and cholesterol.

23. The method of claim 21, wherein said effect on liver biology activity is selected from the group consisting of treating cirrhosis, chronic liver disease, alcoholic liver damage, toxic chemical exposure, NSAID-liver damage, estrogen induced liver problems, bile disorders, and environmental chemical hypersensitivity.

24. The method of claim 21, wherein said effect on heart and artery function is treating or reducing heart and/or artery disease risk due to elevated blood levels of homocysteine.

25. The method of claim 21, wherein said effect on cartilage, bone and joint health is selected from the group consisting of treating osteoarthritis, rheumatoid arthritis, fibromyalgia, joint injuries, joint inflammation, joint degeneration, and osteoporosis.

26. The method of claim 21, wherein said effect on immune function is selected from the group consisting of treating organ transplant rejection, graft rejection, lupus, uveitis, Behcet's disease, Graves disease, Guillain-Barre syndrome, psoriasis, acute dermatomyositis, atopic skin disease, scleroderma, eczema, aplastic anemia, primary cirrhosis, autoimmune hepatitis, ulcerative colitis, Crohn's disease, amyotrophic lateral sclerosis, myasthenia gravis, multiple sclerosis, hepatic syndrome, glomerulonephritis, rheumatoid arthritis, and diabetes mellitus.
